SEQUENCE LISTING (26 sequences)

SEQ ID NO: 1 - DNA, 2217 bp, unidentified
  source 1..2217: mol_type = genomic DNA; note = Adeno-associated virus serotype rh. 10
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 2 - protein, 738 aa, unidentified
  source 1..738: note = Adeno-associated virus serotype rh. 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 3 - protein, 9 aa, unidentified
  source 1..9: note = Adeno-associated virus 2
NLQRGNRQA

SEQ ID NO: 4 - protein, 9 aa, unidentified
  source 1..9: note = Adeno-associated virus 3
NLQSSNTAP

SEQ ID NO: 5 - protein, 9 aa, unidentified
  source 1..9: note = Adeno-associated virus 7
NLQAANTAA

SEQ ID NO: 6 - protein, 9 aa, unidentified
  source 1..9: note = Adeno-associated virus 8
NLQQQNTAP

SEQ ID NO: 7 - protein, 9 aa, unidentified
  source 1..9: note = Adeno-associated virus serotype rh. 10
NLQQQNAAP

SEQ ID NO: 8 - protein, 9 aa, unidentified
  source 1..9: note = Adeno-associated virus 13
NLQNSNAGP

SEQ ID NO: 9 - protein, 9 aa, unidentified
  source 1..9: note = Adeno-associated virus 9
NHQSAQAQA

SEQ ID NO: 10 - protein, 31 aa, unidentified
  source 1..31: note = Adeno-associated virus 1
EVQYTSNYAKSANVDFTVDNNGLYTEPRPIG

SEQ ID NO: 11 - protein, 31 aa, unidentified
  source 1..31: note = Adeno-associated virus 2
EIQYTSNYNKSVNVDFTVDTNGVYSEPRPIG

SEQ ID NO: 12 - protein, 31 aa, unidentified
  source 1..31: note = Adeno-associated virus 5
EIQYTNNYNDPQFVDFAPDSTGEYRTTRPIG

SEQ ID NO: 13 - protein, 31 aa, unidentified
  source 1..31: note = Adeno-associated virus 8
EIQYTSNYYKSTSVDFAVNTEGVYSEPRPIG

SEQ ID NO: 14 - protein, 31 aa, unidentified
  source 1..31: note = Adeno-associated virus 9
EIQYTSNYYKSNNVEFAVNTEGVYSEPRPIG

SEQ ID NO: 15 - protein, 31 aa, unidentified
  source 1..31: note = Adeno-associated virus serotype rh. 10
EIQYTSNYYKSTNVDFAVNTDGTYSEPRPIG

SEQ ID NO: 16 - protein, 14 aa, unidentified
  source 1..14: note = Adeno-associated virus 2
NLQRGNRQAATADV

SEQ ID NO: 17 - protein, 14 aa, unidentified
  source 1..14: note = Adeno-associated virus 3
NLQSSNTAPTTRTV

SEQ ID NO: 18 - protein, 14 aa, unidentified
  source 1..14: note = Adeno-associated virus 7
NLQAANTAAQTQVV

SEQ ID NO: 19 - protein, 14 aa, unidentified
  source 1..14: note = Adeno-associated virus 8
NLQQQNTAPQIGTV

SEQ ID NO: 20 - protein, 14 aa, unidentified
  source 1..14: note = Adeno-associated virus serotype rh. 10
NLQQQNAAPIVGAV

SEQ ID NO: 21 - protein, 14 aa, unidentified
  source 1..14: note = Adeno-associated virus serotype rh. 8
NNQAANTQAQTGLV

SEQ ID NO: 22 - protein, 14 aa, unidentified
  source 1..14: note = Adeno-associated virus 9
NHQSAQAQAQTGWV

SEQ ID NO: 23 - DNA, 2211 bp, unidentified
  source 1..2211: mol_type = genomic DNA; note = Adeno-associated virus 3a
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 24 - protein, 736 aa, unidentified
  source 1..736: note = Adeno-associated virus 3a
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 25 - DNA, 2211 bp, unidentified
  source 1..2211: mol_type = genomic DNA; note = Adeno-associated virus 3b
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 26 - protein, 736 aa, unidentified
  source 1..736: note = Adeno-associated virus 3b
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2023/0074894 A1
(43) Pub. Date: Mar. 9, 2023

(54) AAV VARIANTS WITH HOST ANTIBODY ESCAPE CAPABILITIES AND ALTERED TISSUE TARGETING PROPERTIES

(71) Applicant: University of Florida Research Foundation, Incorporated, Gainesville, FL (US)

(72) Inventors: Mavis Agbandje-McKenna, Gainesville, FL (US); Mario Mietzsch, Gainesville, FL (US)

(73) Assignee: University of Florida Research Foundation, Incorporated, Gainesville, FL (US)

(21) Appl. No.: 18/045,873

(22) Filed: Oct. 12, 2022

Related U.S. Application Data
(63) Continuation-in-part of application No. 17/359,324, filed on Jun. 25, 2021.
Application No. 17/359,324 is a continuation of application No. 16/338,397, filed on Mar. 29, 2019, now Pat. No. 11,078,238.
(60) Provisional application No. 62/401,824, filed on Sep. 29, 2016.
(60) Provisional application No. 63/254,984, filed on Oct. 12, 2021.

(51) Int. Cl. C07K 14/005; C12N 15/86

(52) U.S. Cl. CPC C07K 14/005; C12N 15/86; C12N 2015/8518

(57) ABSTRACT

This disclosure relates to variant AAVrh.10 and AAV3 particles engineered to escape host neutralizing antibodies but retain or improve transduction efficiency, and their use as gene delivery vehicles.

### RELATED APPLICATIONS

This application claims the benefit under 35 U.S.C. § 119(e) of the filing date of U.S. Provisional Application No. 63/254,984, filed Oct. 12, 2021; and is a Continuation-In-Part of U.S. patent application Ser. No. 17/359,324, filed Jun. 25, 2021, which is a continuation of U.S. application Ser. No. 16/338,397, filed Mar. 29, 2019 and issued on Aug. 3, 2021 as U.S. Pat. No. 11,078,238, which is a national stage filing under 35 U.S.C. § 371 of International Patent Application Serial No. PCT/US2017/054600, filed Sep. 29, 2017, which claims the benefit under 35 U.S.C. § 119(e) of U.S. provisional application No. 62/401,824, filed Sep. 29, 2016. The entire contents of each of these applications are incorporated by reference herein.

### GOVERNMENT SUPPORT

This invention was made with government support under R01 GM082946; awarded by the National Institutes of Health. The government has certain rights in the invention.

### SEQUENCE LISTING

In accordance with 37 C.F.R. 1.52(e)(5), the present specification makes reference to a Sequence Listing (submitted electronically as a .xml file named “U119670106U500-SEQ-KSB”). The .xml file was generated on Oct. 12, 2022, and is 37,345 bytes in size. The Sequence Listing is herein incorporated by reference in its entirety.

### BACKGROUND

AAV-based liver-targeted gene therapy—including for the treatment of hemophilia—has been difficult to develop. None of the commonly used AAV serotypes (e.g., AAV2, AAV5, AAV8) have been shown to be efficient at targeting primary human hepatocytes. Additionally, neutralizing antibodies against the respective AAV serotypes are highly prevalent in humans, and, if present, are associated with reduced, or abrogated, therapeutic efficacy. Further, cross-reactivity commonly occurs between variants, such that a neutralizing antibody against a first AAV serotype vector may also neutralize a second AAV serotype vector. When host neutralizing antibodies are encountered, treatment of a disease using AAV particles which are neutralized by the host's neutralizing antibodies can cause detrimental immunogenic effects.

### SUMMARY

The inventors of this disclosure have discovered that certain AAV serotypes (e.g., AAVrh.10, AAV3) cross-react with anti-capsid antibodies against other AAV serotypes (e.g., AAV8) for which there is up to 40% sero-prevalence in the human population. This is likely due to the structural similarity in some surface loops between the neutralizing antibodies (e.g., AAV2, AAV8) and the AAV serotype of choice for a specific treatment (e.g., AAVrh.10, AAV3). Patients who test positive against the AAV serotype of choice for a specific treatment have to be excluded from the cohort.

In order to circumvent the problems of antibody neutralization of certain AAV serotypes with therapeutic promise (e.g., AAVrh.10, AAV3), the inventors of this disclosure have engineered variant recombinant AAVrh.10 and AAV3 (e.g., AAV3b) particles that have one or more mutations at one or more amino acid positions in one or more capsid proteins that enable the variant particles to escape neutralizing antibodies, while retaining (e.g., at least partially), for example without diminishing, or, in some embodiments, improving the transduction efficiency.

Recombinant AAVrh.10 and AAV3 particles disclosed herein can be used in human gene delivery for patients that test positive for neutralizing antibodies against different AAV serotypes (e.g., AAV2, AAV8) who would otherwise be excluded from a treatment comprising AAVrh.10 or AAV3 (e.g., AAV3b) particle-delivered gene therapy (e.g., due to cross-reactivity). The disclosed AAVrh.10 and AAV3 gene therapy particles that escape from pre-existing antibodies against other serotypes provide an alternative serotype for the treatment of patients previously treated with an AAV of another serotype (e.g., AAV2, AAV8). These particles can also be used for treating patients previously treated with an AAV vector of the same serotype (e.g., an AAVrh.10 vector, such as in the late infantile neuronal ceroid lipofuscinosis [AAVrh.10-CUCLN2], metachromatic leukodystrophy [AAVrh.10-hARSA], mucopolysaccharidosis Type IIIA disease [AAVrh.10-SGSH-IRESSUMF1], and hemophilia B [AAVrh.10-FIX] trials).

Use of the disclosed variant AAVrh.10 and AAV3 (e.g., AAV3b) particles that escape pre-existing antibodies in clinical trials has the potential to increase the patient cohort that can be enrolled in clinical trials, thus enabling gene therapy in a much larger percentage of the population.

This disclosure relates, at least in part, to the solution of the structure of AAVrh.10 particles, the structure-guided approach that the inventors took to engineer mutations in the capsid proteins of AAVrh.10, and an analysis of the cross-reactivity of antigenic epitopes. This disclosure is also based on structural mapping of a LacNAc receptor binding site at the icosahedral two-fold axes on the AAVrh.10 capsid surface. The information from this mapping was used to guide the engineering of variant AAVrh.10 particles with mutations which reduce reactivity to neutralizing antibodies while avoiding mutations within the glycan binding interface. Thus, this disclosure is based on structure-based studies related to antigenic epitopes and receptor binding on the capsid to engineer AAVrh.10 particles that escape host neutralizing antibodies, while retaining (e.g., at least partially) or improving transduction efficiency and tissue tropism.

Accordingly, in some aspects, disclosed herein is a recombinant adeno-associated virus rh.10 (rAAVrh.10) particle comprising a capsid protein comprising one or more mutations. The one or more mutations may result in modulated reactivity to a neutralizing antibody and/or altered transduction efficiency of the rAAVrh.10 particle harboring the one or more mutations, relative to a wild-type AAVrh.10 particle. A wild-type AAVrh.10 particle may have a capsid protein with an amino acid sequence of SEQ ID NO: 2.

In some embodiments, a neutralizing antibody is against AAVrh.10. In some embodiments, a neutralizing antibody is against AAV of another serotype. AAV of another serotype may be AAV1, AAV2, AAV3, AAV4, AAV5, AAV6, AAV7, AAV8, AAV9, AAV10, AAV11, AAV12 or AAV13. In some embodiments, AAV of another serotype is AAV8. In some embodiments, a neutralizing antibody is ADK8, ADK9, IVIG, HL2381 or HL2383. In some embodiments, a neutralizing antibody is ADK8. In some embodiments, a neutralizing antibody is ADK8/9. In some embodiments, a neutralizing antibody is HL2381. In some embodiments, a neutralizing antibody is HL2383.

In some embodiments, the reactivity to neutralizing antibodies of a rAAVrh.10 particle comprising a capsid protein with one or more mutations is decreased compared to wild-type AAVrh.10 particles. In some embodiments, the reactivity to neutralizing antibodies is decreased by 5-100% compared to wild-type AAVrh.10 particles. In some embodiments, the reactivity to neutralizing antibodies is decreased by 70-100% compared to wild-type AAVrh.10 particles.

In some embodiments, the transduction efficiency of a rAAVrh.10 particle comprising a capsid protein with one or more mutations is increased compared to wild-type AAVrh.10 particles. In some embodiments, the transduction efficiency is increased by 5-200% compared to wild-type AAVrh.10 particles. In some embodiments, the transduction efficiency is increased by 5-60% compared to wild-type AAVrh.10 particles.

In some embodiments, the transduction efficiency of a rAAVrh.10 particle comprising a capsid protein with one or more mutations is decreased compared to wild type AAVrh.10 particles. In some embodiments, the transduction efficiency is decreased by 5-100% compared to wild type AAVrh.10 particles. In some embodiments, the transduction efficiency is decreased by 20-70% compared to wild type AAVrh.10 particles. In some embodiments, the capsid protein which comprises one or more mutations is one or more of the capsid proteins selected from the group consisting of VP1, VP2 and VP3 (see FIG. 1).

Based on the structure of AAVrh.10 capsid and alignment with AAV8, a mutation is located on a surface loop of the rAAVrh.10 particle. FIG. 3A and Table 1 show the loops on the capsid surface. Accordingly, in some embodiments, a mutation is at one or more amino acid positions selected from the group consisting of: K259, K333, S453, S501, S559, Q589, N590, A592, S671, T674, Y708 and T719. In some embodiments, a mutation may be a substitution (e.g., a conservative amino acid substitution, a substitution with a hydrophobic amino acid, for example A, L, or V, a substitution with a polar amino acid, for example N, S, or Q, or other amino acid substitution) or a deletion. In some embodiments, one or more mutations on a capsid protein of a rAAVrh.10 particle is selected from the group consisting of: K259L, K333V, ΔS453, S501A, S559A, Q589N, N590S, A592Q, S671A, T674V, Y708A and T719V.

In some embodiments, one or more mutations on a capsid protein of a rAAVrh.10 particle is K259L. In some embodiments, one or more mutations on a capsid protein of a rAAVrh.10 particle is ΔS453. In some embodiments, one or more mutations on a capsid protein of a rAAVrh.10 particle is S559A. In some embodiments, one or more mutations on a capsid protein of a rAAVrh.10 particle is S671A. In some embodiments, one or more mutations on a capsid protein of a rAAVrh.10 particle is T719V. In some embodiments, one or more mutations on a capsid protein of a rAAVrh.10 particle are N590S and A592Q. In some embodiments, one or more mutations on a capsid protein of a rAAVrh.10 particle are Q589N, N590S and A592Q. In some embodiments, one or more mutations on a capsid protein of a rAAVrh.10 particle are ΔS453, S559A, Q589N, N590S, A592Q and T719V. In some embodiments, one or more mutations on a capsid protein of a rAAVrh.10 particle is Y708A. In some embodiments, one or more mutations on a capsid protein of a rAAVrh.10 particle are K259L, ΔS453, S559A, Q589N, N590S, A592Q and T719V. In some embodiments, one or more mutations on a capsid protein of a rAAVrh.10 particle is K333V. In some embodiments, one or more mutations on a capsid protein of a rAAVrh.10 particle is S501A. In some embodiments, one or more mutations on a capsid protein of a rAAVrh.10 particle is T674V.

The rAAVrh.10 particles described herein were developed with the purpose of using them as gene delivery vehicles while diminishing the antigenic host response toward the particles. Accordingly, in some embodiments, a rAAVrh.10 particle comprising a capsid protein comprising one or more mutations further comprises a transgene comprising a gene of interest. In some embodiments, a gene of interest encodes a therapeutic protein. A therapeutic protein may be an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.

In some embodiments, a gene of interest encodes a detectable molecule. In some embodiments, a rAAVrh.10 particle comprising a capsid protein comprising one or more mutations further comprises a transgene comprising more than one gene of interest. One gene of interest might encode a therapeutic protein, while another encodes a detectable molecule. In some embodiments, genes of interest comprised in a rAAVrh.10 molecule encode multiple different therapeutic proteins and/or detectable molecules.

In some embodiments, a gene of interest encodes a detectable molecule. A detectable molecule may be a fluorescent protein, a bioluminescent protein, or a protein that provides color, or a fragment thereof.

In some aspects, provided herein is a composition comprising any one of the rAAVrh.10 particles disclosed herein. In some embodiments, a composition of rAAVrh.10 particles further comprises a pharmaceutically acceptable carrier.

In some aspects, provided herein is a method of delivering a protein of interest to a subject, the method comprising administering to the subject a composition comprising any one of the rAAVrh.10 particles disclosed herein that comprise a transgene comprising a gene of interest that encodes the protein of interest.

In some aspects, provided herein is a vector that can be used to make or package any one of the rAAVrh.10 particles of this disclosure. Such a vector may comprise a nucleic acid encoding Cap proteins, wherein the Cap proteins form any one of the rAAVrh.10 particles disclosed herein.

This disclosure also provides other tools useful for the preparation or packaging of rAAVrh.10 particles in the form of a kit. Accordingly, in some aspects, provided herein is a kit comprising any one of the vectors disclosed herein, wherein the vector is contained in container. A kit may further comprise a vector comprising AAV helper genes, wherein the vector comprising the cap gene and the vector comprising AAV helper genes are provided in separate containers. In some embodiments, a kit comprises a vector comprising AAVrh.10 cap gene, a vector comprising helper genes, and packaging cells that are contained in third container. In some embodiments, AAV helper genes encode E1, E2, E4 and/or VA helper proteins.

In some aspects, disclosed herein is a recombinant adeno-associated virus 3 (rAAV3) particle comprising a capsid protein comprising one or more mutations. The one or more mutations may result in modulated reactivity to a neutralizing antibody and/or altered transduction efficiency of the rAAV3 particle harboring the one or more mutations relative to a wild-type AAV3 particle. A wild-type AAV3 particle may have a capsid protein with an amino acid sequence of SEQ ID NO: 24 or SEQ ID NO: 26. In some embodiments, the rAAV3 particle is a rAAV3a particle. In some embodiments, the rAAV3 particle is a rAAV3b particle.

In some embodiments, a neutralizing antibody is against AAV3. In some embodiments, a neutralizing antibody is against AAV of another serotype. AAV of another serotype may be AAV1, AAV2, AAV4, AAV5, AAV6, AAV7, AAV8, AAV9, AAV10, AAVrh.10, AAV11, AAV12, AAV13, or AAVrh.39. In some embodiments, a neutralizing antibody is against AAV2 or AAV8. In some embodiments, a neutralizing antibody is ADK8, IVIG, HL2381 or HL2383. In some embodiments, a neutralizing antibody is ADK8. In some embodiments, a neutralizing antibody is IVIG. In some embodiments, a neutralizing antibody is HL2381. In some embodiments, a neutralizing antibody is HL2383.

In some embodiments, the reactivity to neutralizing antibodies of a rAAV3 particle comprising a capsid protein with one or more mutations is decreased compared to wild-type AAV3 particles. In some embodiments, the reactivity to neutralizing antibodies is decreased by 50-100% compared to wild-type AAV3 particles. In some embodiments, the reactivity to neutralizing antibodies is decreased by 75-100% compared to wild-type AAV3 particles.

In some embodiments, the capsid protein which comprises one or more mutations is one or more of the capsid proteins selected from the group consisting of: VP1, VP2 and VP3.

In some embodiments, the one or more mutations in a rAAV3 particle of the disclosure comprise an amino acid substitution (e.g., a conservative amino acid substitution, a substitution with a hydrophobic amino acid, for example A, L, or V, a substitution with a polar amino acid, for example N, S, or Q, or other amino acid substitution) or an amino acid deletion. In some embodiments, the one or more mutations on a capsid protein of a rAAV3 particle are in an amino acid position(s) selected from the group consisting of: N588, A590, S384, and T717. In some embodiments, the one or more mutations on a capsid protein of a rAAV3 particle are amino acid substitution(s) selected from the group consisting of: N588A, N588S, A590Q, S384A, and T717V. In some embodiments, the one or more mutations on a capsid protein of a rAAV3 particle is N588A. In some embodiments, the one or more mutations on a capsid protein of a rAAV3 particle is N588S. In some embodiments, the one or more mutations on a capsid protein of a rAAV3 particle is A590Q. In some embodiments, the one or more mutations on a capsid protein of a rAAV3 particle is S384A. In some embodiments, the one or more mutations on a capsid protein of a rAAV3 particle is T717V. In some embodiments, the one or more mutations on a capsid protein of a rAAV3 particle are N588A and A590Q. In some embodiments, the one or more mutations on a capsid protein of a rAAV3 particle are N588S and A590Q. In some embodiments, the one or more mutations on a capsid protein of a rAAV3 particle are N588A, A590Q, S384A, and T717V. In some embodiments, the one or more mutations on a capsid protein of a rAAV3 particle are N588S, A590Q, S384A, and T717V.

Aspects of the invention relate to a capsid protein of serotype AAV3 comprising one or more mutations in amino acid positions selected from the group consisting of: N588 and A590 in SEQ ID NO: 24 or SEQ ID NO: 26. In some embodiments, the one or more mutations are amino acid substitution(s) selected from the group consisting of: N588A, N588S, and A590Q. In some embodiments, the one or more mutations are N588A and A590Q. In some embodiments, the one or more mutations are N588S and A590Q.

Aspects of the invention relate to a capsid protein of serotype AAV3 comprising one or more mutations in amino acid positions selected from the group consisting of: N588, A590, S384, and T717 in SEQ ID NO: 24 or SEQ ID NO: 26. In some embodiments, the one or more mutations are amino acid substitution(s) selected from the group consisting of: N588A, N588S, A590Q, S384A, and T717V. In some embodiments, the one or more mutations are N588A, A590Q, S384A, and T717V. In some embodiments, the one or more mutations are N588S, A590Q, S384A, and T717V.

The rAAV3 particles of the disclosure were developed with the purpose of using them as gene delivery vehicles while diminishing the antigenic host response toward the particles. Accordingly, in some embodiments, a rAAV3 particle comprising a capsid protein comprising one or more mutations further comprises a transgene comprising a gene of interest. In some embodiments, a gene of interest encodes a therapeutic protein. A therapeutic protein may be an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.

In some embodiments, a gene of interest encodes a detectable molecule. In some embodiments, a rAAV3 particle comprising a capsid protein comprising one or more mutations further comprises a transgene comprising more than one gene of interest. One gene of interest might encode a therapeutic protein, while another encodes a detectable molecule. In some embodiments, genes of interest comprised in a rAAV3 molecule encode multiple different therapeutic proteins and/or detectable molecules.

In some embodiments, a gene of interest encodes a detectable molecule. A detectable molecule may be a fluorescent protein, a bioluminescent protein, or a protein that provides color, or a fragment thereof.

In some aspects, provided herein is a composition comprising any one of the rAAV3 particles disclosed herein. In some embodiments, a composition of rAAV3 particles further comprises a pharmaceutically acceptable carrier.

In some aspects, provided herein is a method of delivering a protein of interest to a subject, the method comprising administering to the subject a composition comprising any one of the rAAV3 particles disclosed herein that comprise a transgene comprising a gene of interest that encodes a protein of interest (e.g., one or more therapeutic or detectable molecules, as described herein).

In some embodiments, the subject is a mammalian subject. In some embodiments, the mammalian subject is a human, a non-human primate, non-primate mammal, or mouse. In some embodiments, the subject has or is suspected of having a disease or disorder selected from the group consisting of: cancer, diabetes, autoimmune disease, kidney disease, cardiovascular disease, pancreatic disease, intestinal disease, liver disease, hemophilia, α1-antitrypsin (AAT) deficiency, ischemia, skeletal disease and pulmonary disease. In some embodiments, administration of the rAAV3 particle to the subject results in the prevention, alleviation or amelioration of one or more signs or symptoms of the disease or disorder. In some embodiments, the rAAV3 particle is administered to the subject subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs of the subject.

In some aspects, provided herein is a vector that can be used to make or package any one of the rAAV3 particles of this disclosure. Such a vector may comprise a nucleic acid encoding Cap proteins, wherein the Cap proteins form any one of the rAAV3 particles disclosed herein.

This disclosure also provides other tools useful for the preparation or packaging of rAAV3 particles in the form of a kit. Accordingly, in some aspects, provided herein is a kit comprising any one of the vectors disclosed herein, wherein the vector is contained in container. A kit may further comprise a vector comprising AAV helper genes, wherein the vector comprising the cap gene and the vector comprising AAV helper genes are provided in separate containers. In some embodiments, a kit comprises a vector comprising AAV3 cap gene, a vector comprising helper genes, and packaging cells that are contained in third container. In some embodiments, AAV helper genes encode E1, E2, E4 and/or VA helper proteins.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows a depiction of the AAVrh.10 VP proteins encoded by the cap gene.

FIGS. 2A-2B show the AAVrh.10 structure obtained by cryo-electron microscopy (EM) and image reconstruction. FIG. 2A shows an AAVrh.10 micrograph: 0.89 Å/pixel. FIG. 2B shows that AAVrh.10 exhibits the surface topology conserved in all AAVs: depressions at the 2-fold axis, cylindrical channel at the 5-fold axis, and three protrusions around the 3-fold axis.

FIGS. 3A-3C show the structural alignment of AAV8 and AAVrh.10. FIG. 3A shows the structural alignment of VP3 sequence of AAV8 versus the VP3 sequence of AAVrh.10. Capsid surface loops are circled. The VP3 sequence identity is 92.5 percent and the RMSD is 0.65 Å. The structure of VP3 shown is missing the first 15 amino acids at the N terminus. FIG. 3B shows data from an ELISA confirming the footprint of monoclonal antibody ADK8 on AAV8 (Gurda, et al., J Virol. (2012); 86(15):7739-51). FIG. 3C shows AAV8-ADK8 footprint and structure superposition of AAV8 (dark grey) and AAVrh.10 (light grey). The position of ADK8 footprint is shown in a box and enlarged to the right hand side. The amino acid positions are labeled.

FIG. 4 shows antibody recognition of AAV2, AAV8 and AAVrh.10 capsids by native dot blot analysis. Black dots represent recognition while blank regions indicate lack of recognition. A20 was used as a negative control antibody. ADK8, ADK8/9, HL2381, and HL2383 are antibodies developed for AAV8 capsids. All of the AAV8-antibodies cross-reacted with AAVrh.10.

FIGS. 5A-5F show antibody escape phenotypes of AAVrh.10 variants. FIG. 5A depicts AAVrh.10 VP protein mutations that were engineered. FIG. 5B shows the amino acid sequence of the ADK8 epitope in different AAV serotypes. The + and − signs indicate AAV serotypes which can or cannot bind to ADK8, respectively. Grey highlights indicate conserved residues among AAV serotypes while boxed residues indicate residues conserved among ADK8 binders only. FIG. 5C shows the amino acid sequence of the ADK8/9 epitope in different AAV serotypes. The + and − signs indicate AAV serotypes which can or cannot bind to ADK8/9, respectively. Grey highlights indicate conserved residues among AAV serotypes while boxed residues indicate residues conserved among ADK8/9 binders. FIG. 5D shows native dot blot analysis of AAVrh.10 variants probed with the ADK8, ADK8/9, and A20 antibodies. Black dots represent recognition while blank regions indicate lack of recognition. The AAVrh.10 N590S/A592Q variant fully escapes from ADK8 and partially escapes from ADK8/9. The AAV2, AAV3 and AAV13 were used as negative control particles. A20 was used as a negative control antibody. FIG. 5E shows native dot blot analysis of AAVrh.10 variants probed with the ADK8, ADK8/9, and HL2383 antibodies. Black dots represent recognition while blank regions indicate lack of recognition. FIG. 5F shows native dot blot analysis of AAVrh.10 variants probed with the ADK8 and ADK8/9 antibodies. Black dots represent recognition while blank regions indicate lack of recognition. The AAVrh.10 Q589N/N590S/A592Q variant fully escaped from ADK8 and also escaped from ADK8/9.

FIGS. 6A-6B show data from an experiment to identify a receptor that binds to AAVrh.10 (Mietzch, et al., J Virol. (2014); 88(5):2991-3003). FIG. 6A shows that neither heparan sulphate proteoglycans (HSPG), sialic acid nor a terminal galactose is an AAVrh.10 receptor. FIG. 6B shows data from a glycan array showing hits for AAV5 but no hits for AAVrh.10.

FIGS. 7A-7B show another analysis of the Consortium for Functional Glycomics (CFG) glycan array data. FIG. 7A shows a more magnified view of the data depicted in FIG. 6B and the glycans that were identified using a lower threshold. FIG. 7B depicts particular glycans that were identified as binding to AAVrh.10.

FIGS. 8A-8B show screening of AAVs on a LacNAc glycan array. FIG. 8A shows that AAVrh.10 binds to sulfated N-acetyllactosamine (LacNAc). FIG. 8B shows that 6S N-Acetyl-glucosamine is required for binding of AAVrh.10 to LacNAc.

FIGS. 9A-9B show identification of the glycan binding site on AAVrh.10 capsid by cryo-EM reconstruction. FIG. 9A shows a cryo-EM reconstruction at 4.3 Å of AAVrh.10 capsid complexed to LacNAc glycan molecules (#6, ˜1 kDa and 100 molecules per VP monomer). FIG. 9B shows the difference map obtained by subtraction of a AAVrh.10 density map from a glycan-containing AAVrh.10 density map, which revealed additional density indicated by the boxed regions at the surface of the 2-fold symmetry axes.

FIGS. 10A-10C show identification of glycan contact residues. FIG. 10A shows amino acid residues at the glycan binding site. FIG. 10B shows sequence conservation in AAV of various serotypes at the binding site. FIG. 10C shows transduction efficiency of wild-type and AAVrh.10 Y708A mutant particles in cells transduced with wild-type or variant/mutant AABrh.10 comprising luciferase.

FIG. 11 shows transduction efficiency of engineered AAVrh.10 variants measured based on luciferase expression. Variants showed increased transduction efficiency compared to wild-type (WT) AAVrh.10.

FIG. 12 shows observed antibody escape and transduction efficiency phenotypes for AAVrh.10 variants.

FIG. 13 shows predicted antibody escape and transduction efficiency phenotypes for AAVrh.10 variants.

FIG. 14 provides alignment of non-limiting examples of AAVrh.10 mutations with AAV of other serotypes.

FIGS. 15A-15E show determination of the antigenicity of the AAVrh.10 capsid. FIG. 15A shows immune-dot blot analysis of native rAAV capsids of indicated serotypes. 10¹⁰ genome-containing particles were spotted on a nitrocellulose membrane. The membranes were incubated with a panel of mAbs as depicted to the left of the membrane. Monoclonal antibody B1 served as an internal loading control. FIGS. 15B and 15C show sequence alignments for the VR-VIII loop of a selection of AAV serotypes (SEQ ID NOs: 16-22, from top to bottom, respectively). Amino acids highlighted in light grey indicate sequence identity among the AAVs. In addition amino acids highlighted in darker grey indicate common residues (FIG. 15B) among the ADK8 binding AAV serotypes in the ADK8 binding epitope and among the ADK8/9 binding AAV serotypes in the ADK8/9 binding epitope (FIG. 15C). FIG. 15D shows the relative positions of seven mutations (7× mut) introduced into the VP proteins of AAVrh.10.

FIG. 15E shows a comparison between the transduction of AAVrh.10 wild-type vectors and the generated AAVrh.10 7× mutant in HEK 293 cells by a luciferase assay (MOI 100,000).

FIGS. 16A-16D show prevention of antibody-mediated AAVrh.10 neutralization. FIGS. 16A and 16B show neutralization assays using increasing amounts of purified monoclonal antibodies, ADK8 (FIG. 16A) and ADK8/9 (FIG. 16B), with purified AAVrh.10 wild-type (dark grey) or 7× mut (light grey) vectors carrying a luciferase gene (MOI 100,000). FIG. 16C shows a similar neutralization assay as in (FIG. 16A), except that different human serum samples were used instead of monoclonal antibodies. FIG. 16D shows native dot blot analysis using the human serum samples used in (FIG. 16C) as primary antibodies on AAV8 and AAVrh.10.

FIG. 17 shows cross-reactivity between conformation-specific monoclonal antibodies raised against the AAV8 capsid, the AAV2 capsid and the AAV3b capsid, implying structural conservation of some epitopes between the capsid serotypes.

FIGS. 18A-18C show pseudoatomic models of virus-Fab binding. FIG. 18A shows a 3D cryo-reconstruction of purified AAV3b wild-type capsids in complex with purified ADK8. FIG. 18B shows a 3D cryo-reconstruction of purified AAV3b wild-type capsids in complex with purified HL2381. FIG. 18C shows a 3D cryo-reconstruction of purified AAV3b wild-type capsids in complex with purified HL2383.

FIGS. 19A-19B show the effect of neutralizing antibodies on AAV3b single capsid variants. FIG. 19A shows a dot blot demonstrating that three anti-AAV8 antibodies have reduced recognition of the AAV3b capsid variants, relative to wild-type AAV3b. Black dots represent recognition while blank regions indicate lack of recognition. FIG. 19B shows a neutralization assay demonstrating that the infectivity of the AAV3b capsid variants in presence of neutralizing antibodies showed a slight reduction of transgene expression for all samples.

FIGS. 20A-20B show the purity and titer of an AAV3b variant of the disclosure (NSAQ) relative to wild-type AAV3b. FIG. 20A shows wild-type AAV3b. FIG. 20B shows an AAV3b variant of the disclosure (NSAQ).

FIG. 21 shows dot blots demonstrating that an AAV3b variant of the disclosure (NSAQ) escaped monoclonal antibody detection. Dot blots were performed to verify antibody binding to the wild-type AAV3b capsid and determine if AAV3b variant of the disclosure (NSAQ) was capable of antibody evasion. Black dots represent recognition while blank regions indicate lack of recognition.

FIGS. 22A-22D show that an AAV3b variant of the disclosure (NSAQ) transduced cells in the presence of neutralizing antibodies. FIG. 22A shows that an AAV3b variant of the disclosure (NSAQ) showed similar transduction efficiency to a wild-type AAV3b capsid. FIG. 22B shows the transduction efficiency of an AAV3b variant of the disclosure (NSAQ) relative to a wild-type AAV3b capsid for the antibody ADK8. FIG. 22C shows the transduction efficiency of an AAV3b variant of the disclosure (NSAQ) relative to a wild-type AAV3b capsid for the antibody HL2381. FIG. 22D shows the transduction efficiency of an AAV3b variant of the disclosure (NSAQ) relative to a wild-type AAV3b capsid for the antibody HL2383.

FIG. 23 shows observed antibody escape and transduction efficiency phenotypes for AAV3 variants.

### DETAILED DESCRIPTION

### AAVrh.10 Particles

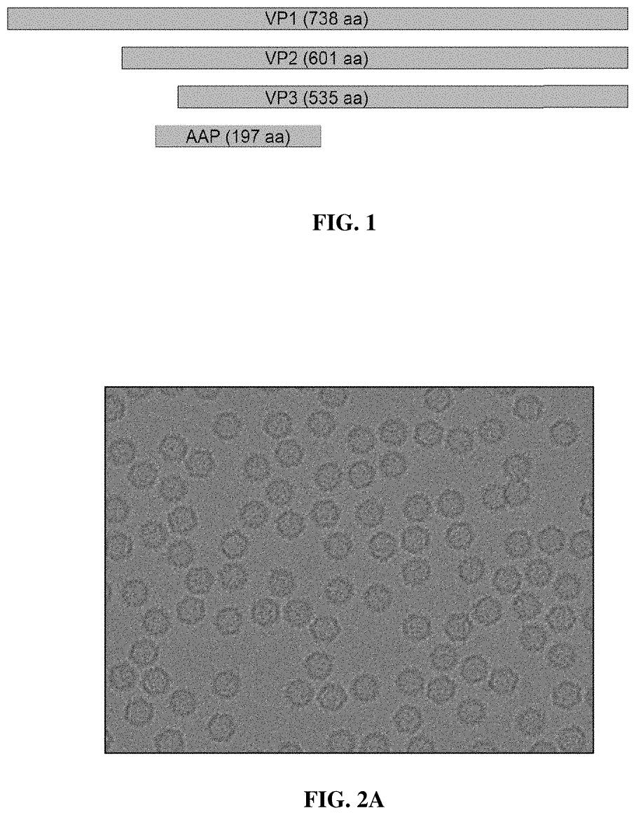

Not very much is known about the structure of AAVrh.10 particles. What is known is that AAVrh.10 belongs to clade E and is closely related to AAV8. The cap gene of AAVrh.10 is 89.2% identical to the nucleotide sequence of the cap gene of AAV8, while the encoded VP1 protein of AAVrh.10 shares 93.5% identity (and 96.2% similarity) to the VP1 protein of AAV8. Further, AAVrh.10 is not the same as AAV10. There is a 12 amino acid difference between AAVrh.10 and AAV10. FIG. 1 shows the proteins encoded by the AAVrh.10 cap gene. The AAVrh.10 cap gene encodes the capsid proteins VP1, VP2 and VP3, and assembly-activating protein (AAP). No more information about AAVrh.10 particle structure was known prior to the work carried out by the inventors of this disclosure.

To address the problem of AAVrh.10 particles reacting to host neutralizing antibodies against AAVrh.10 and AAV particles of other serotypes, the inventors first solved the AAVrh.10 capsid structure by cryo-reconstruction, and determined antibody cross-reactivity between AAVrh.10 and antibodies directed to other AAV serotypes. The inventors also identified a glycan receptor involved in the cell transduction by AAVrh.10 as well as the receptor binding site on the AAVrh.10 capsid (see Examples below for data). This information was then utilized in a structure based approach, which was not possible before the work done by the inventors, to engineer mutations in the AAVrh.10 capsid that enable the variant AAVrh.10 particles to escape binding to neutralizing antibodies either without altering transduction efficiency and tissue targeting, or, in some embodiments, with improved transduction efficiency.

### Engineered AAVrh.10 Particles

Disclosed herein is a recombinant AAVrh.10 (rAAVrh.10) particle comprising a capsid protein comprising one or more mutations that result in modulated reactivity to neutralizing antibodies. In some embodiments, a variant recombinant AAVrh.10 particle as disclosed herein comprises mutations that result in altered transduction efficiency. In some embodiments, mutations in the capsid protein of AAVrh.10 particles results in both modulated reactivity to neutralizing antibodies and altered transduction efficiency. In some embodiments, mutations in the capsid protein of AAVrh.10 particles results in decreased reactivity to neutralizing antibodies and unaltered transduction efficiency, relative to wild-type AAVrh.10 particles. In some embodiments, mutations in the capsid protein of AAVrh.10 particles results in decreased reactivity to neutralizing antibodies and increased transduction efficiency, relative to wild-type AAVrh.10 particles.

As described herein, variant AAVrh.10 particles are those that have an amino acid sequence that is different from the sequence of wild-type AAVrh.10 particles. The term “engineered” is used synonymously with the term “recombinant.” The term “variant” as used herein means different from wild-type. A wild-type AAVrh.10 particle may be one found in nature or a recombinant viral particle that is made in a laboratory setting for the purpose of testing phenotypes. A variant AAVrh.10 particle as used herein is one that is engineered.

In some embodiments, the difference in amino acid sequence is present in one or more capsid proteins (e.g., VP1, VP2, VP3, VP1 and VP2, VP1 and VP3, or VP1, VP2 and VP3). The AAV genome encodes overlapping sequences of the three capsid proteins, VP1, VP2 and VP3, which starts from one promoter. All three of the proteins are translated from one mRNA. After the mRNA is synthesized, it can be spliced in different ways, resulting in expression of the three proteins VP1, VP2 and VP3 (FIG. 1). The difference between the amino acid sequence between a “variant” AAVrh.10 particle and a wild-type particle may be in one or more amino acids. For example, a variant AAVrh.10 may contain only 1 or 2, 3, 4, 5, 6, or 7 or more mutations compared to the amino acid sequence of wild-type AAVrh.10. The nucleic acid encoding and the amino acid sequence of wild-type AAVrh.10 capsid proteins are provided as SEQ ID NOs: 1 and 2, respectively. Table 1 provides amino acid sequence limitations for the VP1, VP2 and VP3 capsid proteins.

Nucleic acid sequence of the cap gene for wild-type AAVrh.10
(SEQ ID NO: 1)
ATGGCTGCCGATGGTTATCTTCCAGATTGGCTCGAGGACAACCTCTCTGAGGGCATTCGCGAGT
GGTGGGACTTGAAACCTGGAGCCCCGAAACCCAAAGCCAACCAGCAAAAGCAGGACGACGGCCG
GGGTCTGGTGCTTCCTGGCTACAAGTACCTCGGACCCTTCAACGGACTCGACAAGGGGGAGCCC
GTCAACGCGGCGGACGCAGCGGCCCTCGAGCACGACAAGGCCTACGACCAGCAGCTCAAAGCGG
GTGACAATCCGTACCTGCGGTATAACCACGCCGACGCCGAGTTTCAGGAGCGTCTGCAAGAAGA
TACGTCTTTTGGGGGCAACCTCGGGCGAGCAGTCTTCCAGGCCAAGAAGCGGGTTCTCGAACCT
CTCGGTCTGGTTGAGGAAGGCGCTAAGACGGCTCCTGGAAAGAAGAGACCGGTAGAGCCATCAC
CCCAGCGTTCTCCAGACTCCTCTACGGGCATCGGCAAGAAAGGCCAGCAGCCCGCGAAAAAGAG
ACTCAACTTTGGGCAGACTGGCGACTCAGAGTCAGTGCCCGACCCTCAACCAATCGGAGAACCC
CCCGCAGGCCCCTCTGGTCTGGGATCTGGTACAATGGCTGCAGGCGGTGGCGCTCCAATGGCAG
ACAATAACGAAGGCGCCGACGGAGTGGGTAGTTCCTCAGGAAATTGGCATTGCGATTCCACATG
GCTGGGCGACAGAGTCATCACCACCAGCACCCGAACCTGGGCCCTCCCCACCTACAACAACCAC
CTCTACAAGCAAATCTCCAACGGGACTTCGGGAGGAAGCACCAACGACAACACCTACTTCGGCT
ACAGCACCCCCTGGGGGTATTTTGACTTTAACAGATTCCACTGCCACTTCTCACCACGTGACTG
GCAGCGACTCATCAACAACAACTGGGGATTCCGGCCCAAGAGACTCAACTTCAAGCTCTTCAAC
ATCCAGGTCAAGGAGGTCACGCAGAATGAAGGCACCAAGACCATCGCCAATAACCTTACCAGCA
CGATTCAGGTCTTTACGGACTCGGAATACCAGCTCCCGTACGTCCTCGGCTCTGCGCACCAGGG
CTGCCTGCCTCCGTTCCCGGCGGACGTCTTCATGATTCCTCAGTACGGGTACCTGACTCTGAAC
AATGGCAGTCAGGCCGTGGGCCGTTCCTCCTTCTACTGCCTGGAGTACTTTCCTTCTCAAATGC
TGAGAACGGGCAACAACTTTGAGTTCAGCTACCAGTTTGAGGACGTGCCTTTTCACAGCAGCTA
CGCGCACAGCCAAAGCCTGGACCGGCTGATGAACCCCCTCATCGACCAGTACCTGTACTACCTG
TCTCGGACTCAGTCCACGGGAGGTACCGCAGGAACTCAGCAGTTGCTATTTTCTCAGGCCGGGC
CTAATAACATGTCGGCTCAGGCCAAAAACTGGCTACCCGGGCCCTGCTACCGGCAGCAACGCGT
CTCCACGACACTGTCGCAAAATAACAACAGCAACTTTGCCTGGACCGGTGCCACCAAGTATCAT
CTGAATGGCAGAGACTCTCTGGTAAATCCCGGTGTCGCTATGGCAACCCACAAGGACGACGAAG
AGCGATTTTTTCCGTCCAGCGGAGTCTTAATGTTTGGGAAACAGGGAGCTGGAAAAGACAACGT
GGACTATAGCAGCGTTATGCTAACCAGTGAGGAAGAAATTAAAACCACCAACCCAGTGGCCACA
GAACAGTACGGCGTGGTGGCCGATAACCTGCAACAGCAAAACGCCGCTCCTATTGTAGGGGCCG
TCAACAGTCAAGGAGCCTTACCTGGCATGGTCTGGCAGAACCGGGACGTGTACCTGCAGGGTCC
TATCTGGGCCAAGATTCCTCACACGGACGGAAACTTTCATCCCTCGCCGCTGATGGGAGGCTTT
GGACTGAAACACCCGCCTCCTCAGATCCTGATTAAGAATACACCTGTTCCCGCGGATCCTCCAA
CTACCTTCAGTCAAGCTAAGCTGGCGTCGTTCATCACGCAGTACAGCACCGGACAGGTCAGCGT
GGAAATTGAATGGGAGCTGCAGAAAGAAAACAGCAAACGCTGGAACCCAGAGATTCAATACACT
TCCAACTACTACAAATCTACAAATGTGGACTTTGCTGTTAACACAGATGGCACTTATTCTGAGC
CTCGCCCCATCGGCACCCGTTACCTCACCCGTAATCTGTAA
Amino acid sequence of the capsid protein for wild-type AAVrh.10
(SEQ ID NO: 2)
MAADGYLPDWLEDNLSEGIREWWDLKPGAPKPKANQQKQDDGRGLVLPGYKYLGPFNGLDKGEP
VNAADAAALEHDKAYDQQLKAGDNPYLRYNHADAEFQERLQEDTSFGGNLGRAVFQAKKRVLEP
LGLVEEGAKTAPGKKRPVEPSPQRSPDSSTGIGKKGQQPAKKRLNFGQTGDSESVPDPQPIGEP
PAGPSGLGSGTMAAGGGAPMADNNEGADGVGSSSGNWHCDSTWLGDRVITTSTRTWALPTYNNH
LYKQISNGTSGGSTNDNTYFGYSTPWGYFDFNRFHCHFSPRDWQRLINNNWGFRPKRLNFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFEFSYQFEDVPFHSSYAHSQSLDRLMNPLIDQYLYYL
SRTQSTGGTAGTQQLLFSQAGPNNMSAQAKNWLPGPCYRQQRVSTTLSQNNNSNFAWTGATKYH
LNGRDSLVNPGVAMATHKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKIPHTDGNFHPSPLMGGF
GLKHPPPQILIKNTPVPADPPTTFSQAKLASFITQYSTGQVSVEIEWELQKENSKRWNPEIQYT
SNYYKSTNVDFAVNTDGTYSEPRPIGTRYLTRNL

An AAVrh.10 particle may be an empty capsid, or an AAV particle comprising one or more nucleic acids.

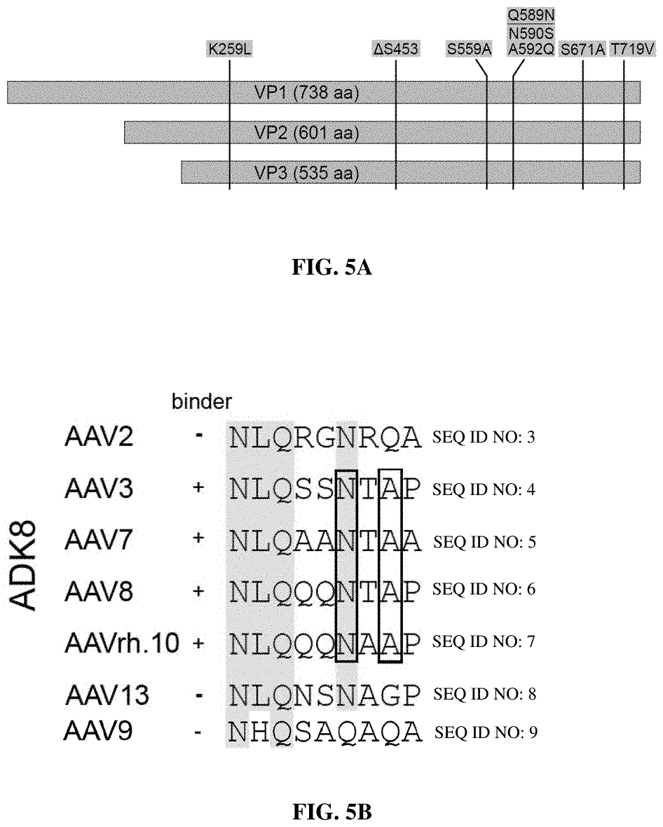

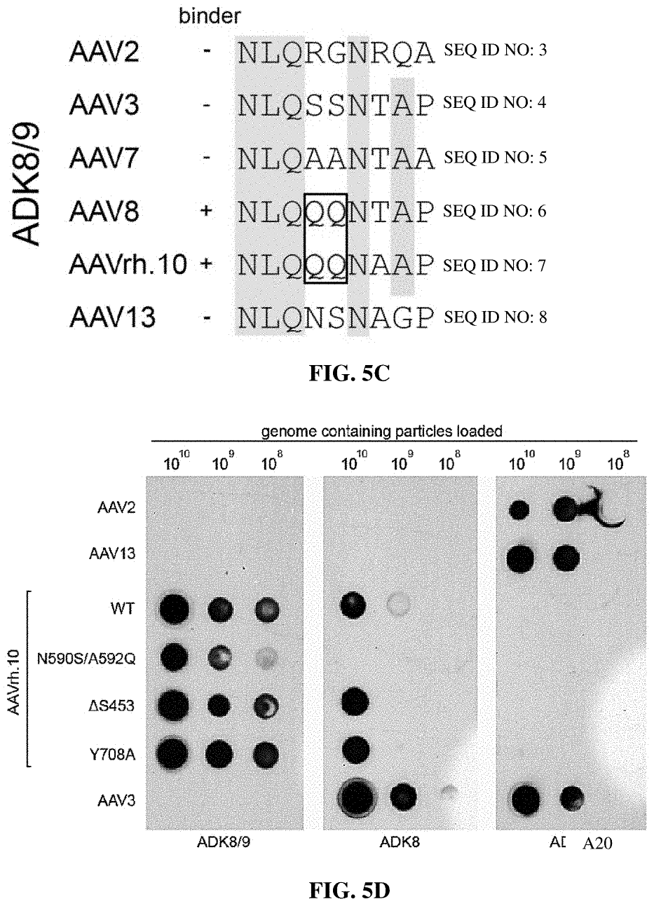

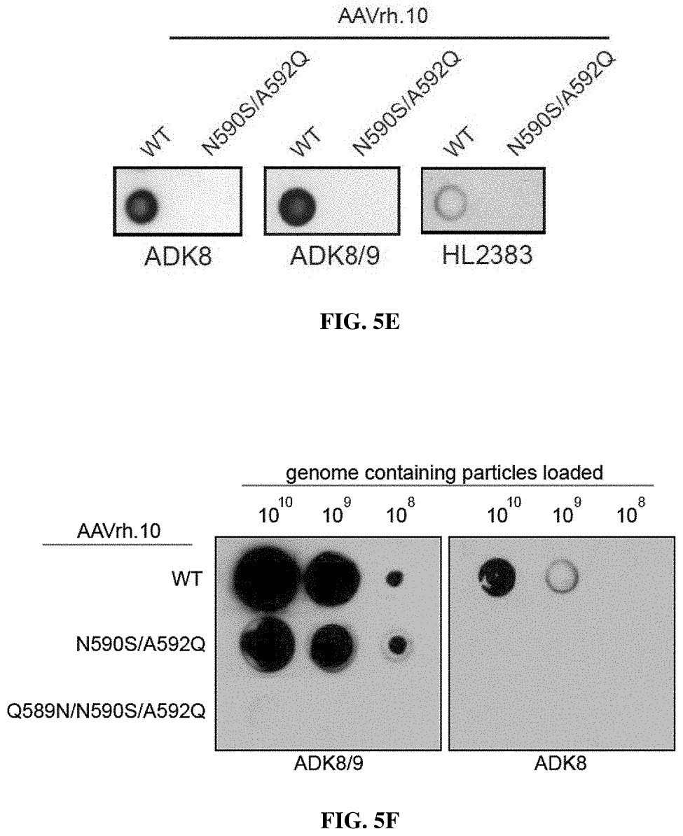

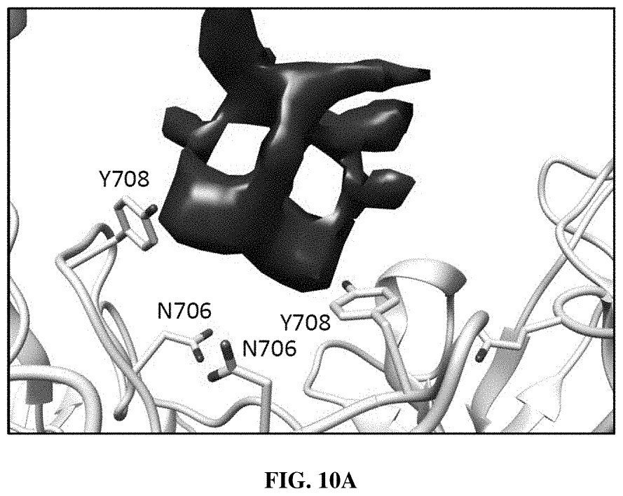

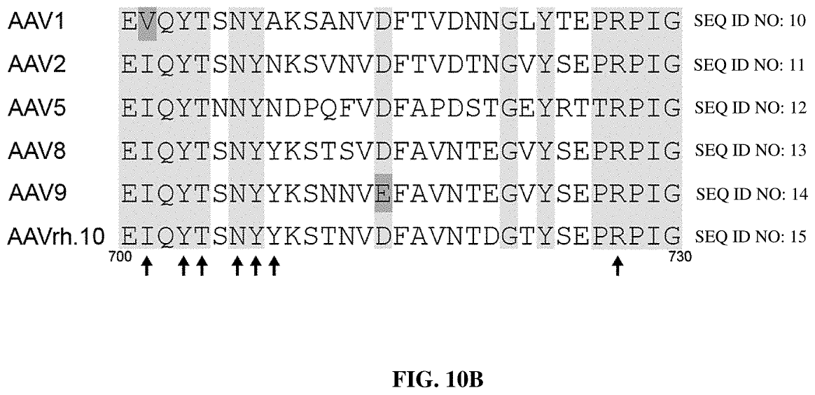

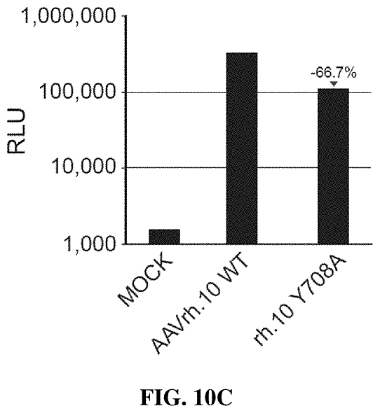

In some embodiments, a mutation in a capsid protein of a variant recombinant AAVrh.10 particle is an amino acid deletion (e.g., ΔS453). In some embodiments, a mutation is an amino acid substitution. In some embodiments, a substitution comprises an amino acid that is not present or conserved in one or more AAV particles of another serotype (e.g., AAV1, AAV2, AAV3, AAV4, AAV5, AAV6, AAV7, AAV8, AAV9, AAV10, AAV11, AAV12 or AAV13). In some embodiments, the other serotype may be an another AAV isolated from rhesus macaque tissue that is other than AAVrh.10. Gao, et al. (PNAS (2003); 100(10):6081-6086) discloses 37 capsid clones isolated from rhesus macaque tissues, and is incorporated herein by reference in its entirety. Non-limiting examples of conserved amino acid residues can be found in FIG. 5B, FIG. 5C and FIG. 10B. Sequences of other AAV serotypes are known and can be aligned with SEQ ID NO: 2 (wild-type AAVrh.10 capsid protein) using techniques known in the art. In some embodiments, a substituted amino acid is not hydrophobic in nature.

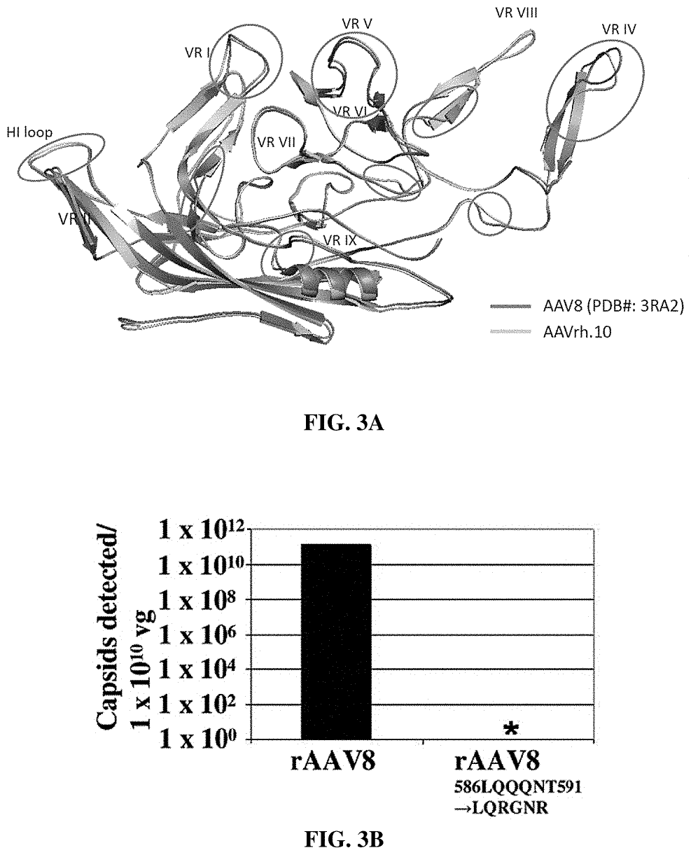

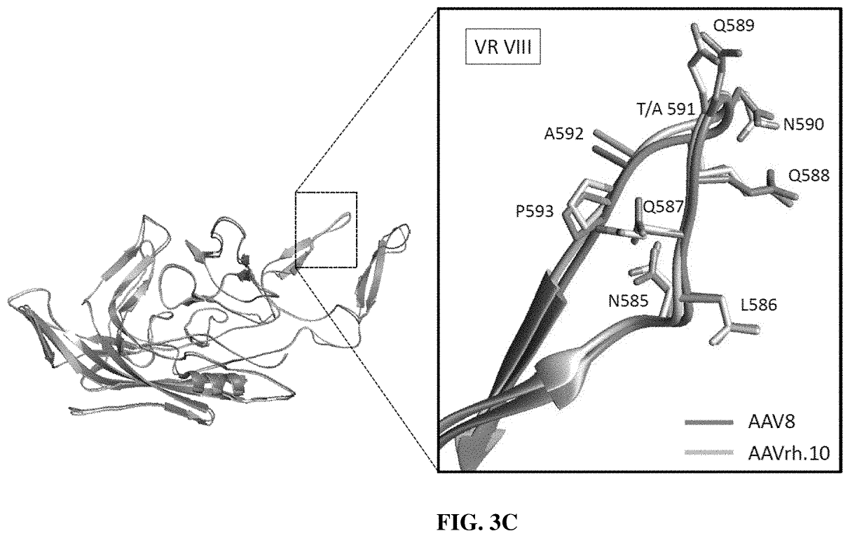

In some embodiments, a mutation in variant recombinant AAVrh.10 is on a capsid surface loop as depicted in FIG. 3A. A mutation in a variant rAAVrh.10 particle may be in any one of the surface loops depicted in FIG. 3A. The amino acid definitions of these loops are listed in Table 1.

TABLE 1
Amino add definition of AAVrh.10
capsid protein regions and loops

| AAVrh.10 VP regions/loops | Amino acid definition |
|---|---|
| VP1 complete | 1-738 |
| VP1u | 1-137 |
| VP1/2 common region | 138-203 |
| VP3 | 204-738 |
| βB | 239-251 |
| βC | 280-287 |
| βD | 307-327 |
| βE | 343-349 |
| βF | 403-408 |
| βG | 410-419 |
| βH | 648-654 |
| βI | 676-691 |
| VR I | 263-271 |
| VR II | 329-333 |
| VR III | 383-391 |
| VR IV | 452-471 |
| VR V | 490-507 |
| VR VI | 528-544 |
| VR VII | 547-559 |
| VR VIII | 582-597 |
| HI-looρ | 655-675 |
| VR IX | 707-714 |

In some embodiments, a mutation is at any one of the amino acid positions selected from the group consisting of: K259, K333, S453, S501, S559, Q589, N590, A592, S671, T674, Y708 and T719 of AAVrh.10.

Non-limiting examples of mutations at these amino acid positions are K259L, K333V, ΔS453, S501A, S559A, Q589N, N590S, A592Q, S671A, T674V, Y708A and T719V.

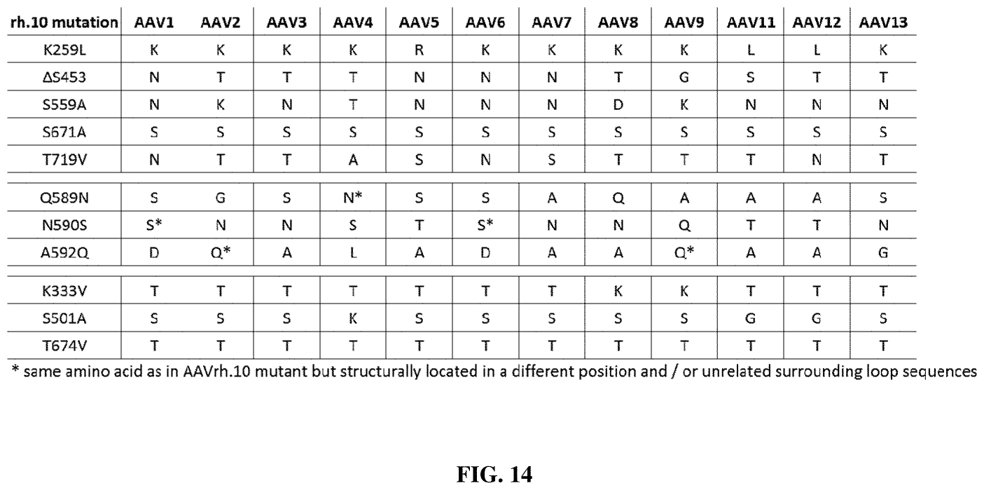

In some embodiments, mutations comprise substitutions with amino acid residues that are not found on AAV of other serotypes. FIG. 14 provides amino acid residues in AAV particles of other serotypes in comparison to non-limiting examples of AAVrh.10 mutations.

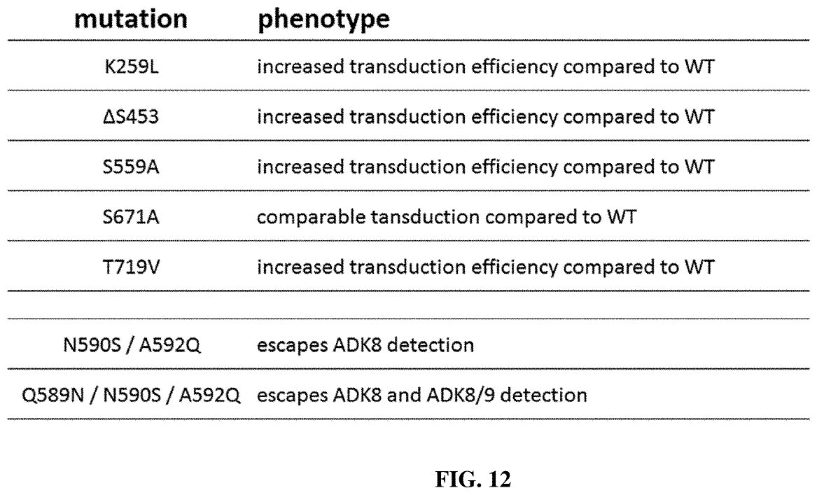

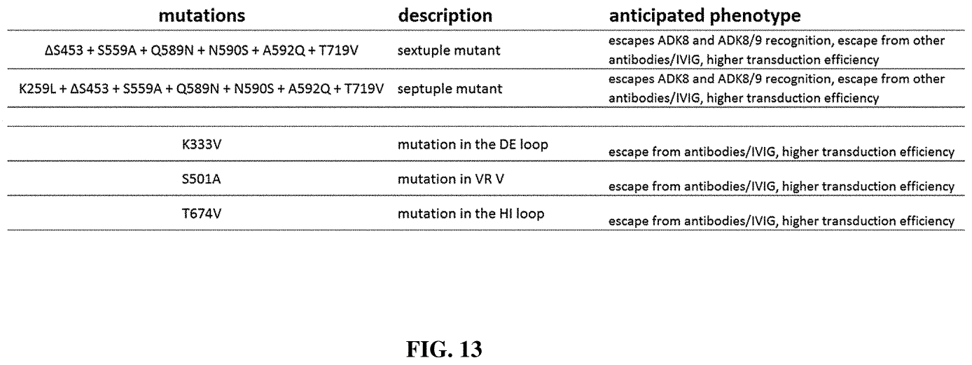

In some embodiments, more than one mutation is present on a recombinant or engineered AAVrh.10 capsid (e.g., 2, 3, 4, 5, 6, or 7 or more) mutations. Non-limiting examples of AAVrh.10 particles with multiple mutations are provided in FIG. 12 and FIG. 13. It is to be understood that any one mutation provided in this disclosure can be combined with any other mutation provided herein. It is also to be understood that AAVrh.10 variants with multiple mutations provided herein are merely examples and are non-limiting. Any one of the mutations disclosed in any one variant AAVrh.10 particle having multiple mutations may exist as a single mutation in an engineered AAVrh.10 particle, or in combination with one or more of the other mutations disclosed here. For example, a recombinant AAVrh.10 particle as disclosed herein may contain any one of the mutations K259L, K333V, ΔS453, S501A, S559A, Q589N, N590S, A592Q, S671A, T674V, Y708A, or T719V. A recombinant AAVrh.10 particle as disclosed herein may contain more than one of any of the mutations K259L, K333V, ΔS453, S501A, S559A, Q589N, N590S, A592Q, S671A, T674V, Y708A, or T719V. In some embodiments, a recombinant AAVrh.10 particle as disclosed herein comprises the mutations N590S and A592Q. In some embodiments, a recombinant AAVrh.10 particle as disclosed herein comprises the mutations Q589N, N590S and A592Q. In some embodiments, a recombinant AAVrh.10 particle as disclosed herein comprises the mutations ΔS453, S559A, Q589N, N590S, A592Q and T719V. In some embodiments, a recombinant AAVrh.10 particle as disclosed herein comprises the mutations K259L, ΔS453, S559A, Q589N, N590S, A592Q and T719V. In some embodiments, a recombinant AAVrh.10 particle as disclosed herein comprises the mutations Q589N, N590S, A592Q and T719V. FIG. 12 lists some non-limiting examples of variant AAVrh.10 particles and their observed phenotypes. FIG. 13 lists some non-limiting examples of variant AAVrh.10 particles and their predicted phenotypes.

### Cross Reactivity of AAVrh.10 to Neutralizing Antibodies

A neutralizing antibody is an antibody that defends a cell from an antigen or infectious body by neutralizing any effect it has biologically. In some embodiments, a neutralizing antibody to which an engineered variant AAVrh.10 particle shows modulated reactivity compared to a wild-type AAVrh.10 particle may be any neutralizing antibody that reacts to any AAV particle. In some embodiments, a neutralizing antibody is against AAVrh.10. In some embodiments, a neutralizing antibody is against an AAV particle of another serotype (e.g., AAV1, AAV2, AAV3, AAV4, AAV5, AAV6, AAV7, AAV8, AAV9, AAV10, AAV11, AAV12 or AAV13). In some embodiments, the other serotype may be an another AAV isolated from rhesus macaque tissue that is other than AAVrh.10. In some embodiments, an AAV of another serotype is AAV2, AAV3, AAV7, AAV8, AAV9 or AAV13. In some embodiments, an AAV of another serotype is AAV8.

In some embodiments, a neutralizing antibody to which an engineered variant AAVrh.10 particle shows modulated reactivity compared to wild-type AAVrh.10 reacts to AAVs of more than one serotype (e.g., an antibody reacting to both AAV8 and AAV9). In some embodiments, an AAV other than AAVrh.10 is a hybrid AAV and comprises sequences from more than one AAV serotype.

In some embodiments, a neutralizing antibody is ADK8, ADK9, IVIG, HL2381 or HL2383.

In some embodiments, a mutation in a capsid protein of any one of the recombinant AAVrh.10 particles disclosed herein results in decreased reactivity to neutralizing antibodies compared to a wild-type AAVrh.10 particle. In some embodiments, reactivity to neutralizing antibodies is decreased by 5-100% (e.g., 5-10, 10-20, 20-40, 40-60, 60-80, 80-90, 90-95, 95-98, 95-99, 95-99.9, 100, 10-100, 30-100, 60-100, 70-100 or 80-100%) compared to wild-type AAVrh.10 particles.

Non-limiting examples of AAVrh.10 capsid protein mutations that result in antibody escape or decreased reactivity to neutralizing antibodies are provided in FIG. 12. Non-limiting examples of AAVrh.10 capsid protein mutations that are predicted to result in antibody escape or decreased reactivity to neutralizing antibodies are provided in FIG. 13. Further non-limiting examples can be found in the Examples.

Reactivity of a recombinant AAVrh.10 particle to a neutralizing antibody can be measured in one of numerous methods known in the art for measuring binding of a virus particle to a protein (e.g., dot blotting, surface plasmon resonance or biolayer interferometry). Neutralization of virus infectivity by antibodies is described by Klasse (Adv Biol. (2014); 2014: 157895).

### Alteration of Transduction Efficiency by Mutations in a AAVrh.10 Capsid Protein

In some embodiments, one or more mutations in a capsid protein of an AAVrh.10 particle results in altered transduction efficiency. The term “transduction” as used herein refers to the entry of an AAV particle into a cell, and is equivalent to “infection” of a cell.

In some embodiments, the transduction efficiency of a variant AAVrh.10 particle as described herein is increased compared to the transduction efficiency of a wild-type AAVrh.10 particle. In some embodiments, the transduction efficiency of a variant AAVrh.10 particle is increased 1.2-500 fold (e.g., 1.2-2, 1.5-3, 3-5, 5-10, 10-100, 100-250 or 200-500 fold) compared to the transduction efficiency of a wild-type AAVrh.10 particle for the same type of cell. In some embodiments, the transduction efficiency of a variant AAVrh.10 particle is increased by 5-200% (e.g., 5-10, 10-30, 20-50, 50-100, 5-60 or 100-200%) compared to the transduction efficiency of a wild-type AAVrh.10 particle for the same type of cell. Mutations in an AAVrh.10 capsid protein that result in increased transduction efficiency are shown in FIG. 12. Mutations in an AAVrh.10 capsid protein that are predicted to result in increased transduction efficiency are shown in FIG. 13.

In some embodiments, the transduction efficiency of a variant AAVrh.10 particle is decreased compared to the transduction efficiency of a wild-type AAVrh.10 particle. In some embodiments, the transduction efficiency of a variant AAVrh.10 particle is decreased 1.2-500 fold (e.g., 1.2-2, 1.5-3, 3-5, 5-10, 10-100, 100-250 or 200-500 fold) compared to the transduction efficiency of a wild-type AAVrh.10 particle for the same type of cell. In some embodiments, the transduction efficiency of a variant AAVrh.10 particle is decreased by 5-100% (e.g., 5-100, 5-10, 10-30, 20-50, 20-70, 50-100, 5-60, 20-80 or 80-100%) compared to the transduction efficiency of a wild-type AAVrh.10 particle for the same type of cell. In some embodiments, a mutation that leads to decreased transduction efficiency is at position Y708 of the AAVrh.10 capsid protein. In some embodiments, a mutation at position Y708 of the AAVrh.10 capsid protein is Y708A.

In some embodiments, a mutation in a AAVrh.10 capsid protein does not alter the transduction efficiency of the variant AAVrh.10 particle harboring the mutation compared to the transduction efficiency of a wild-type AAVrh.10 particle. For example, FIG. 12 shows that a S671A mutation does not change the transduction efficiency of the variant AAVrh.10 particle harboring the mutation compared to the transduction efficiency of a wild-type AAVrh.10 particle.

### AAV3 Particles

Adeno-associated virus serotype 3 (AAV3) is closely related to AAV2, and, like AAV2, is derived from human sources. Compared to vectors based on other AAV serotypes, AAV3 vectors inefficiently transduce most cell types. However, AAV3 vectors have been shown to effectively transduce cochlear inner hair cells (see, e.g., Chang, et al., Prostaglandins Other Lipid Mediat. (2005); 76(1-4):48-58) and human liver cancer cells (see, e.g., Glushakova, et al., Mol Genet Metab. (2009); 98(3):289-99).

Two distinct isolates of AAV3 have been cloned, which differ by only 6 amino acids (Muramatsu, et al., Virol. (1996); 221(1):208-17; Rutledge, et al., Virol. (1998); 72:309-319). The 6 amino acids which differ are located at positions 144, 145, 321, 322, 594, and 598 of the respective capsid proteins. The two isolates have been termed AAV3a and AAV3b. It is hypothesized that the 6 amino acids which differ between AAV3a and AAV3b may be due to errors in the sequencing of the isolates, rather than being genetic differences between AAV3a and AAV3b. As used herein, “AAV3” is used to describe both AAV3a and AAV3b.

Because aspects of the present disclosure relate to capsid-modified AAV3 variants, wild-type reference sequences are provided for the positions at which amino acid mutation(s) may be made to generate the rAAV3 particles of the disclosure. Those reference sequences are the amino acid sequences of the wild-type AAV3a capsid protein (SEQ ID NO: 24) and the wild-type AAV3b capsid protein (SEQ ID NO: 26).

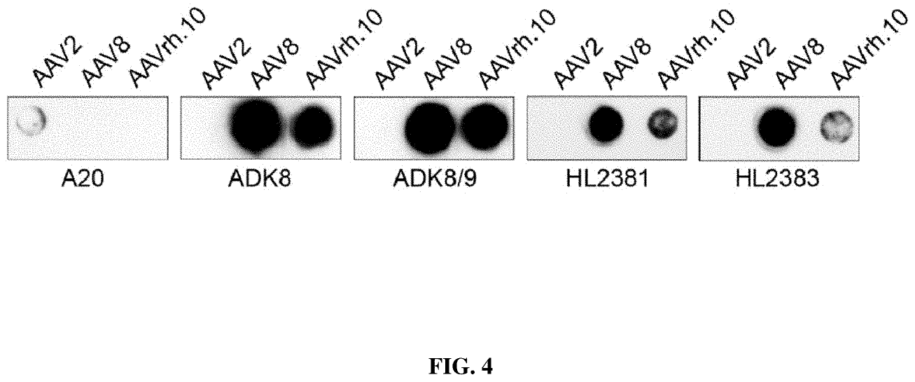

As described above, the capsid proteins (e.g., VP1, VP2, VP3) for AAV3a and AAV3b are not identical (for an alignment, see FIG. 4B of Rutledge, et al., Virol. (1998); 72:309-319). However, it is specifically noted that the amino acids which were altered to create the rAAV3 particles of the disclosure (e.g., N588, A590, S384, and T717) do not differ between the AAV3a and AAV3b capsid proteins (e.g., the amino acid at position 588 is asparagine (N) in both the AAV3a and AAV3b capsid proteins; the amino acid at position 590 is alanine (A) in both the AAV3a and AAV3b capsid proteins; etc.). Therefore, the disclosure embraces both rAAV3a and rAAV3b particles which comprise or consist of the amino acid mutations described herein.

### Engineered AAV3 Particles

Disclosed herein is a recombinant AAV3 (rAAV3) particle comprising a capsid protein comprising one or more mutations that result in modulated reactivity to neutralizing antibodies compared to the reactivity to neutralizing antibodies of a wild-type AAV3 particle. In some embodiments, a variant rAAV3 particle as disclosed herein comprises mutations that result in altered transduction efficiency compared to the transduction efficiency of a wild-type AAV3 particle. In some embodiments, mutations in the capsid protein of AAV3 particles results in both modulated reactivity to neutralizing antibodies and altered transduction efficiency. In some embodiments, mutations in the capsid protein of AAV3 particles results in decreased reactivity to neutralizing antibodies and increased transduction efficiency, compared to a wild-type AAV3 particle. In some embodiments, mutations in the capsid protein of AAV3 particles results in decreased reactivity to neutralizing antibodies and unaltered transduction efficiency, compared to a wild-type AAV3 particle.

As described herein, variant rAAV3 particles are those that have an amino acid sequence that is different from the sequence of wild-type AAV3 particles. The term “engineered” is used synonymously with the term “recombinant.” The term “variant” as used herein means different from wild-type. A wild-type AAV3 particle may be one found in nature or a recombinant viral particle that is made in a laboratory setting for the purpose of testing phenotypes. A variant AAV3 particle as used herein is one that is engineered.

In some embodiments, the difference in amino acid sequence is present in one or more capsid proteins (e.g., VP1, VP2, and VP3; VP1 and VP2; or VP1 and VP3). The AAV genome encodes overlapping sequences of the three capsid proteins, VP1, VP2 and VP3, which starts from one promoter. All three of the proteins are translated from one mRNA. After the mRNA is synthesized, it can be spliced in different ways, resulting in expression of the three proteins VP1, VP2 and VP3. The difference between the amino acid sequence between a “variant” AAV3 particle and a wild-type particle may be in one or more amino acids. For example, a variant AAV3 may contain 1 or 2, 3, 4, 5, 6, or 7 (or more) mutations compared to the amino acid sequence of wild-type AAV3. In some embodiments, a variant AAV3 comprises one or more mutations at an amino acid(s) position which is conserved between the AAV3a and AAV3b capsid sequences (SEQ ID NOs: 24 and 26, respectively). By “conserved” it is meant that the amino acid corresponding to a specific position with the capsid sequences, once aligned, is the same in both the AAV3a and AAV3b capsid sequences (SEQ ID NOs: 24 and 26, respectively). The amino acids which are conserved the AAV3a and AAV3b capsid sequences (SEQ ID NOs: 24 and 26, respectively) are located at positions 1-143, 146-320, 323-593, 595-597, and 599-736. In some embodiments, a variant AAV3 does not comprise one or more mutations at an amino acid(s) position which is not conserved between the AAV3a and AAV3b capsid sequences (SEQ ID NOs: 24 and 26, respectively). The amino acids which are not conserved the AAV3a and AAV3b capsid sequences (SEQ ID NOs: 24 and 26, respectively) are located at positions 144, 145, 321, 322, 594, and 598.

The nucleic acid encoding a wild-type AAV3a capsid protein is provided as SEQ ID NO: 23. The amino acid sequence of a wild-type AAV3a capsid protein is provided as SEQ ID NO: 24. The nucleic acid encoding a wild-type AAV3b capsid protein is provided as SEQ ID NO: 25. The amino acid sequence of a wild-type AAV3b capsid protein is provided as SEQ ID NO: 26.

Nucleic acid sequence of the cap gene for wild-type AAV3a
(SEQ ID NO: 23)
atggctgctgacggttatcttccagattggctcgaggacaacctttctgaaggcattcgtgagt
ggtgggctctgaaacctggagtccctcaacccaaagcgaaccaacaacaccaggacaaccgtcg
gggtcttgtgcttccgggttacaaatacctcggacccggtaacggactcgacaaaggagagccg
gtcaacgaggcggacgcggcagccctcgaacacgacaaagcttacgaccagcagctcaaggccg
gtgacaacccgtacctcaagtacaaccacgccgacgccgagtttcaggagcgtcttcaagaaga
tacgtcttttgggggcaaccttggcagagcagtcttccaggccaaaaagaggatccttgagcct
cttggtctggttgaggaagcagctaaaacggctcctggaaagaagggggctgtagatcagtctc
ctcaggaaccggactcatcatctggtgttggcaaatcgggcaaacagcctgccagaaaaagact
aaatttcggtcagactggagactcagagtcagtcccagaccctcaacctctcggagaaccacca
gcagcccccacaagtttgggatctaatacaatggcttcaggcggtggcgcaccaatggcagaca
ataacgagggtgccgatggagtgggtaattcctcaggaaattggcattgcgattcccaatggct
gggcgacagagtcatcaccaccagcaccagaacctgggccctgcccacttacaacaaccatctc
tacaagcaaatctccagccaatcaggagcttcaaacgacaaccactactttggctacagcaccc
cttgggggtattttgactttaacagattccactgccacttctcaccacgtgactggcagcgact
cattaacaacaactggggattccggcccaagaaactcagcttcaagctcttcaacatccaagtt
agaggggtcacgcagaacgatggcacgacgactattgccaataaccttaccagcacggttcaag
tgtttacggactcggagtatcagctcccgtacgtgctcgggtcggcgcaccaaggctgtctccc
gccgtttccagcggacgtcttcatggtccctcagtatggatacctcaccctgaacaacggaagt
caagcggtgggacgctcatccttttactgcctggagtacttcccttcgcagatgctaaggactg
gaaataacttccaattcagctataccttcgaggatgtaccttttcacagcagctacgctcacag
ccagagtttggatcgcttgatgaatcctcttattgatcagtatctgtactacctgaacagaacg
caaggaacaacctctggaacaaccaaccaatcacggctgctttttagccaggctgggcctcagt
ctatgtctttgcaggccagaaattggctacctgggccctgctaccggcaacagagactttcaaa
gactgctaacgacaacaacaacagtaactttccttggacagcggccagcaaatatcatctcaat
ggccgcgactcgctggtgaatccaggaccagctatggccagtcacaaggacgatgaagaaaaat
ttttccctatgcacggcaatctaatatttggcaaagaagggacaacggcaagtaacgcagaatt
agataatgtaatgattacggatgaagaagagattcgtaccaccaatcctgtggcaacagagcag
tatggaactgtggcaaataacttgcagagctcaaatacagctcccacgactggaactgtcaatc
atcagggggccttacctggcatggtgtggcaagatcgtgacgtgtaccttcaaggacctatctg
ggcaaagattcctcacacggatggacactttcatccttctoctetgatgggaggctttggactg
aaacatccgcctcctcaaatcatgatcaaaaatactccggtaccggcaaatcctccgacgactt
tcagcccggccaagtttgcttcatttatcactcagtactccactggacaggtcagcgtggaaat
tgagtgggagctacagaaagaaaacagcaaacgttggaatccagagattcagtacacttccaac
tacaacaagtctgttaatgtggactttactgtagacactaatggtgtttatagtgaacctcgcc
ctattggaacccggtatctcacacgaaacttgtga
Amino acid sequence of the capsid protein for wild-type AAV3a
(SEQ ID NO: 24)
MAADGYLPDWLEDNLSEGIREWWALKPGVPQPKANQQHQDNRRGLVLPGYKYLGPGNGLDKGEP
VNEADAAALEHDKAYDQQLKAGDNPYLKYNHADAEFQERLQEDTSFGGNLGRAVFQAKKRILEP
LGLVEEAAKTAPGKKGAVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
RGVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTGTVNHQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRNL
Nucleic acid sequence of the cap gene for wild-type AAV3b
(SEQ ID NO: 25)
atggctgctgacggttatcttccagattggctcgaggacaacctttctgaaggcattcgtgagt
ggtgggctctgaaacctggagtccctcaacccaaagcgaaccaacaacaccaggacaaccgtcg
gggtcttgtgettccgggttacaaatacctcggacccggtaacggactcgacaaaggagagccg
gtcaacgaggcggacgcggcagccctcgaacacgacaaagcttacgaccagcagctcaaggccg
gtgacaacccgtacctcaagtacaaccacgccgacgccgagtttcaggagcgtcttcaagaaga
tacgtcttttgggggcaaccttggcagagcagtcttccaggccaaaaagaggatccttgagcct
cttggtctggttgaggaagcagctaaaacggctcctggaaagaagaggcctgtagatcagtctc
ctcaggaaccggactcatcatctggtgttggcaaatcgggcaaacagcctgccagaaaaagact
aaatttcggtcagactggcgactcagagtcagtcccagaccctcaacctctcggagaaccacca
gcagcccccacaagtttgggatctaatacaatggcttcaggcggtggcgcaccaatggcagaca
ataacgagggtgccgatggagtgggtaattcctcaggaaattggcattgcgattcccaatggct
gggcgacagagtcatcaccaccagcaccagaacctgggccctgcccacttacaacaaccatctc
tacaagcaaatctccagccaatcaggagcttcaaacgacaaccactactttggctacagcaccc
cttgggggtattttgactttaacagattccactgccacttctcaccacgtgactggcagcgact
cattaacaacaactggggattccggcccaagaaactcagcttcaagctcttcaacatccaagtt
aaagaggtcacgcagaacgatggcacgacgactattgccaataaccttaccagcacggttcaag
tgtttacggactcggagtatcagctcccgtacgtgctcgggtcggcgcaccaaggctgtctccc
gccgtttccagcggacgtcttcatggtccctcagtatggatacctcaccctgaacaacggaagt
caagcggtgggacgctcatccttttactgcctggagtacttcccttcgcagatgctaaggactg
gaaataacttccaattcagctataccttcgaggatgtaccttttcacagcagctacgctcacag
ccagagtttggatcgcttgatgaatcctcttattgatcagtatctgtactacctgaacagaacg
caaggaacaacctctggaacaaccaaccaatcacggctgctttttagccaggctgggcctcagt
ctatgtctttgcaggccagaaattggctacctgggccctgctaccggcaacagagactttcaaa
gactgctaacgacaacaacaacagtaactttccttggacagcggccagcaaatatcatctcaat
ggccgcgactcgctggtgaatccaggaccagctatggccagtcacaaggacgatgaagaaaaat
ttttccctatgcacggcaatctaatatttggcaaagaagggacaacggcaagtaacgcagaatt
agataatgtaatgattacggatgaagaagagattcgtaccaccaatcctgtggcaacagagcag
tatggaactgtggcaaataacttgcagagctcaaatacagctcccacgactagaactgtcaatg
atcagggggccttacctggcatggtgtggcaagatcgtgacgtgtaccttcaaggacctatctg
ggcaaagattcctcacacggatggacactttcatccttctoctetgatgggaggctttggactg
aaacatccgcctcctcaaatcatgatcaaaaatactccggtaccggcaaatcctccgacgactt
tcagcccggccaagtttgcttcatttatcactcagtactccactggacaggtcagcgtggaaat
tgagtgggagctacagaaagaaaacagcaaacgttggaatccagagattcagtacacttccaac
tacaacaagtctgttaatgtggactttactgtagacactaatggtgtttatagtgaacctcgcc
ctattggaacccggtatctcacacgaaacttgtaa
Amino acid sequence of the capsid protein for wild-type AAV3b
(SEQ ID NO: 26)
MAADGYLPDWLEDNLSEGIREWWALKPGVPQPKANQQHQDNRRGLVLPGYKYLGPGNGLDKGEP
VNEADAAALEHDKAYDQQLKAGDNPYLKYNHADAEFQERLQEDTSFGGNLGRAVFQAKKRILEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRNL

An AAV3 particle may be an empty capsid, or an AAV particle comprising one or more nucleic acids.

In some embodiments, a mutation in a capsid protein of a variant recombinant AAV3 particle is an amino acid deletion. In some embodiments, a mutation is an amino acid substitution. In some embodiments, a substitution comprises an amino acid that is not present or conserved in one or more AAV particles of another serotype (e.g., AAV1, AAV2, AAV4, AAV5, AAV6, AAV7, AAV8, AAV9, AAV10, AAVrh.10, AAV11, AAV12, AAV13, or AAVrh.39). In some embodiments, the other serotype may be an another AAV isolated from rhesus macaque tissue that is other than AAVrh.10 or AAVrh.39. Sequences of other AAV serotypes are known and can be aligned with SEQ ID NO: 24 (wild-type AAV3a capsid protein) or SEQ ID NO: 26 (wild-type AAV3b capsid protein) using techniques known in the art. In some embodiments, a substituted amino acid is not hydrophobic in nature.

In some embodiments, a mutation is at any one of the amino acid positions selected from the group consisting of: N588, A590, S384, and T717. Non-limiting examples of mutations at these amino acid positions are N588A, N588S, A590Q, S384A, and T717V. In some embodiments, mutations comprise substitutions with amino acid residues that are not found on AAV of other serotypes.

In some embodiments, more than one mutations are present on a recombinant or engineered AAV3 capsid (e.g., 1, 2, 3, 4, 5, 6, or 7 or more) mutations. It is to be understood that any one mutation provided in this disclosure can be combined with any other mutation provided herein. It is also to be understood that AAV3 variants with multiple mutations provided herein are merely examples and are non-limiting. Any one of the mutations disclosed in any one variant AAV3 particle having multiple mutations may exist as a single mutation in an engineered AAV3 particle, or in combination with one or more of the other mutations disclosed here. For example, a recombinant AAV3 particle as disclosed herein may contain any one of the mutations N588A, N588S, A590Q, S384A, and T717V. A recombinant AAV3 particle as disclosed herein may contain more than one of any of the mutations N588A, N588S, A590Q, S384A, and T717V. In some embodiments, a recombinant AAV3 particle as disclosed herein comprises the mutation N588A. In some embodiments, a recombinant AAV3 particle as disclosed herein comprises the mutation N588S. In some embodiments, a recombinant AAV3 particle as disclosed herein comprises the mutation S384A. In some embodiments, a recombinant AAV3 particle as disclosed herein comprises the mutation T717V. In some embodiments, a recombinant AAV3 particle as disclosed herein comprises the mutations N588A and A590Q. In some embodiments, a recombinant AAV3 particle as disclosed herein comprises the mutations N588S and A590Q. In some embodiments, a recombinant AAV3 particle as disclosed herein comprises the mutations N588A, A590Q, S384A, and T717V. In some embodiments, a recombinant AAV3 particle as disclosed herein comprises the mutations N588S, A590Q, S384A, and T717V.

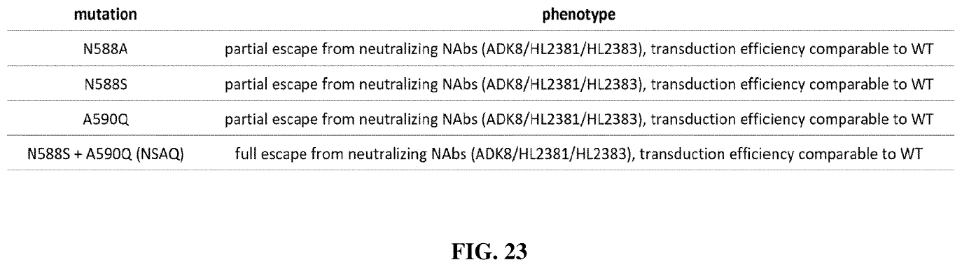

FIG. 23 lists some non-limiting examples of variant AAV3 particles and their observed phenotypes.

### Cross Reactivity of AAV3 to Neutralizing Antibodies

A neutralizing antibody is an antibody that defends a cell from an antigen or infectious body by neutralizing any effect it has biologically. In some embodiments, a neutralizing antibody to which an engineered variant AAV3 particle shows modulated reactivity compared to a wild-type AAV3 particle may be any neutralizing antibody that reacts to any AAV particle. In some embodiments, a neutralizing antibody is against AAV3. In some embodiments, a neutralizing antibody is against an AAV particle of another serotype (e.g., AAV1, AAV2, AAV4, AAV5, AAV6, AAV7, AAV8, AAV9, AAV10, AAVrh.10, AAV11, AAV12, AAV13, or AAVrh.39). In some embodiments, a neutralizing antibody is against another AAV isolated from rhesus macaque tissue that is other than AAVrh.10 or AAVrh.39. In some embodiments, a neutralizing antibody is against an AAV2 or AAV8 particle.

In some embodiments, a neutralizing antibody to which an engineered variant AAV3 particle shows modulated reactivity compared to wild-type AAV3 reacts to AAVs of more than one serotype (e.g., an antibody reacting to both AAV2 and AAV8). In some embodiments, a neutralizing antibody to which an engineered variant AAV3 particle shows modulated reactivity is a hybrid AAV and comprises sequences from more than one AAV serotype.

In some embodiments, a neutralizing antibody is ADK8, IVIG, HL2381 or HL2383. In some embodiments, a neutralizing antibody is ADK8. In some embodiments, a neutralizing antibody is IVIG. In some embodiments, a neutralizing antibody is HL2381. In some embodiments, a neutralizing antibody is HL2383.

In some embodiments, a mutation in a capsid protein of any one of the recombinant AAV3 particles disclosed herein results in decreased reactivity to neutralizing antibodies compared to a wild-type AAV3 particle. In some embodiments, reactivity to neutralizing antibodies is decreased is decreased 1.2-500 fold (e.g., 1.2-2, 1.5-3, 3-5, 5-10, 10-100, 100-250 or 200-500 fold) compared to wild-type AAV3 particles. In some embodiments, reactivity to neutralizing antibodies is decreased by 5-100% (e.g., 5-10, 10-20, 20-40, 40-60, 60-80, 80-90, 90-95, 95-98, 95-99, 95-99.9, 100, 10-100, 30-100, 50-100, 60-100, 70-100 or 80-100%) compared to wild-type AAV3 particles. In some embodiments, reactivity to neutralizing antibodies is decreased by 50-100% (e.g., 50-60, 50-70, 50-80, 65-80, 70-90, 60-90, 75-95, or 85-100%) compared to wild-type AAV3 particles. In some embodiments, reactivity to neutralizing antibodies is decreased by 75-100% (e.g., 75-80, 77-90, 80-93, 85-97, 90-100, or 95-100%) compared to wild-type AAV3 particles.

Non-limiting examples of AAV3 capsid protein mutations that result in antibody escape or decreased reactivity to neutralizing antibodies are provided in FIG. 23. Further non-limiting examples can be found in the Examples.

Reactivity of a recombinant AAV3 particle to a neutralizing antibody can be measured in one of numerous methods known in the art for measuring binding of a virus particle to a protein (e.g., dot blotting, surface plasmon resonance or biolayer interferometry). Neutralization of virus infectivity by antibodies is described by Klasse (Adv Biol. (2014); 2014: 157895).

### Alteration of Transduction Efficiency by Mutations in a AAV3 Capsid Protein

In some embodiments, one or more mutations in a capsid protein of an AAV3 particle results in altered transduction efficiency. The term “transduction” as used herein refers to the entry of an AAV particle into a cell, and is equivalent to “infection” of a cell.

In some embodiments, the transduction efficiency is increased compared to the transduction efficiency of a wild-type AAV3 particle. In some embodiments, the transduction efficiency of a variant AAV3 particle is increased 1.2-500 fold (e.g., 1.2-2, 1.5-3, 3-5, 5-10, 10-100, 100-250 or 200-500 fold) compared to the transduction efficiency of a wild-type AAV3 particle for the same type of cell. In some embodiments, the transduction efficiency of a variant AAV3 particle is increased by 150-400% (e.g., 150-200, 150-300, 200-350, 250-400, 150-360 or 300-400%) compared to the transduction efficiency of a wild-type AAV3 particle for the same type of cell. Mutations in an AAV3 capsid protein that do not result in a decrease in transduction efficiency, relative to a wild-type AAV3 capsid protein, are shown in FIG. 23.

In some embodiments, a mutation in a AAV3 capsid protein does not alter the transduction efficiency of the variant AAV3 particle harboring the mutation (e.g. wild-type transduction efficiency is maintained).

### Recombinant AAV Particles Comprising Recombinant Nucleic Acids

In some embodiments, a recombinant AAVrh.10 or AAV3 particle as disclosed herein is an empty capsid. In some embodiments, any one of the recombinant AAVrh.10 or AAV3 particles disclosed herein comprises a recombinant nucleic acid (e.g., a rAAV genome). In some embodiments, the recombinant nucleic acid comprises a transgene. In some embodiments, the transgene is operably linked or operably connected to a promoter. Accordingly, in some embodiments, a rAAVrh.10 or rAAV3 particle comprises a transgene encoding a gene of interest, wherein the transgene is encapsidated by the viral capsid.

In some embodiments, a gene of interest encodes a therapeutic protein. A therapeutic protein may be an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, or an anticoagulant. Some non-limiting examples of a therapeutic protein are CUCLN2, hARSA and factor IX.

In some embodiments, a gene of interest encodes a detectable molecule. In some embodiments, a detectable molecule is a fluorescent protein, a bioluminescent protein, or a protein that provides color (e.g., β-galactosidase, β-lactamasses, β-glucuronidase and spheriodenone). In some embodiments, a detectable molecule is a fluorescent, bioluminescent or enzymatic protein or functional peptide or functional polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or functional peptides or polypeptides thereof. A blue fluorescent protein may be azurite, EBFP, EBFP2, mTagBFP, or Y66H. A cyan fluorescent protein may be ECFP, AmCyan1, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, and ZsGreen). A yellow fluorescent protein may be EYFP, mBanana, mCitrine, PhiYFp, TagYFP, Topaz, Venus, YPet, or ZsYellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRaspberry, mRFP1, mRuby or mStrawberry.

In some embodiments, a detectable molecule is a bioluminescent protein, or functional peptide or polypeptide thereof. Non-limiting examples of bioluminescent proteins are firefly luciferase, click-beetle luciferase, Renilla luciferase, or luciferase from Oplophorus gracilirostris.

In some embodiments, a detectable molecule may be any polypeptide or protein that can be detected using methods known in the art. Non-limiting methods of detection are fluorescence imaging, luminescent imaging and bright field imaging.

In some embodiments, a nucleic acid is provided, the nucleic acid comprising an expression construct containing a promoter operably linked to a coding sequence of a gene of interest. In some embodiments, a promoter is a natural promoter. In some embodiments, a promoter can be a truncated natural promoter. In some embodiments, a promoter can include an enhancer and/or basal promoter elements from a natural promoter. In some embodiments, a promoter can be or include elements from a CMV, a chicken beta actin, a desmin, or any other suitable promoter or combination thereof. In some embodiments, a promoter can be an engineered promoter. In some embodiments, a promoter is transcriptionally active in host cells. In some embodiments, a promoter is less than 1.6 kb in length, less than 1.5 kb in length, less than 1.4 kb in length, less than 1.3 kb in length, less than 1.2 kb in length, less than 1.1 kb in length, less than 1 kb in length, or less than 900 kb in length.

A promoter is “operably linked” to a nucleotide sequence when the promoter sequence controls and/or regulates the transcription of the nucleotide sequence. A promoter may be a constitutive promoter, tissue-specific promoter, an inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A cytomegalovirus (CMV) promoters. Non-limiting examples of constitutive mammalian promoters include various housekeeping gene promoters, as exemplified by the β-actin promoter (e.g., chicken β-actin promoter) and human elongation factor-1 α (EF-1α) promoter. In some embodiments, chimeric viral/mammalian promoters may include a chimeric CMV/chicken beta actin (CBA, CB or CAG) promoters.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Several promoters are publicly available or described. For example, Ple155 promoter is available through Addgene plasmid repository (Addgene plasmid # 29011) and is described in Scalabrino, et al. (Hum Mol Genet. (2015), 24(21):6229-39). Ye, et al. (Hum Gene Ther. (2016); 27(1):72-82) describes a shorter version of this promoter called PR1.7. A Thyl promoter construct is also available through Addgene plasmid repository (Addgene plasmid #20736). A GRM6 promoter construct is also available through Addgene plasmid repository (Addgene plasmid #66391). Guziewicz, et al. (PLoS One (2013); 8(10):e75666) and Esumi, et al. (J Biol Chem. (2004), 279(18):19064-73) provide examples of the use of VMD2 promoter. Dyka, et al. (Adv Exp Med Biol. (2014); 801: 695-701) describes cone-specific promoters for use in gene therapy, including IRBP and IRBPe-GNAT2 promoter. The use of PR2.1 promoter has been demonstrated in Komáromy, et al. (Gene Ther. (2008); 15(14):1049-55) and its characterization in Karim, et al. (Tree Physiol. (2015); 35(10):1129-39). Aartsen, et al. (PLoS One, (2010); 5(8):e12387) describes the use of GFAP promoter to drive GFP expression in Muller glial cells. Other examples of Muller glia specific promoters are RLBP1 and GLAST (Vázquez-Chona, Invest Ophthalmol Vis Sci. (2009), 50(8):3996-4003; Regan, et al., J Neurosci, (2007), 27(25): 6607-6619).

Synthetic promoters are also contemplated herein. A synthetic promoter may comprise, for example, regions of known promoters, regulatory elements, transcription factor binding sites, enhancer elements, repressor elements, and the like.

It is to be understood that a promoter may be a fragment of any one of the promoters disclosed herein, or one that retains partial promoter activity (e.g., 10-90, 30-60, 50-80, 80-99 or 90-99.9% of the activity) of a whole promoter.

In some embodiments, an AAV genome is built of single-stranded deoxyribonucleic acid (ssDNA), which is either positive- or negative-sensed. At each end of the DNA strand is an inverted terminal repeat (ITR). Between the ITRs are two open reading frames (ORFs): rep and cap. The rep ORF is composed of four overlapping genes encoding Rep proteins required for the AAV life cycle. The cap ORF contains overlapping nucleotide sequences of capsid proteins: VP1, VP2 and VP3, which interact together to form a capsid of an icosahedral symmetry.

In some embodiments, the nucleic acid encoding the gene of interest (e.g., along with a promoter) can be inserted in between two ITRs (e.g., by replacing one or more of the viral genes). Accordingly, in some embodiments, a gene of interest is flanked by inverted terminal repeats (ITRs), e.g., AAV ITRs, ITRs from AAVrh.10, AAV3, or any other AAV serotype including but not limited to AAV2, AAV4, AAV5, AAV6, AAV7, AAV8, or AAV9. In some embodiments, a recombinant rAAV particle comprises a nucleic acid vector, such as a single-stranded (ss) or self-complementary (sc) AAV nucleic acid vector. In some embodiments, the nucleic acid vector contains an expression construct as described herein and one or more regions comprising inverted terminal repeat (ITR) sequences (e.g., wild-type ITR sequences or engineered ITR sequences) flanking the expression construct. In some embodiments, the nucleic acid is encapsidated by a viral capsid.

### Variants

Aspects of the disclosure relate to recombinant AAV3 and AAVrh.10 capsid proteins. Variants of the capsid proteins described in this application are also encompassed by this disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between. In some embodiments, the disclosure provides variants of an AAVrh.10 capsid protein. In some embodiments, the disclosure provides variants of an AAV3 capsid protein.

Unless otherwise noted, the term “sequence identity,” as known in the art, refers to a relationship between the sequences of two polypeptides (or polynucleotides), as determined by sequence comparison (alignment). In some embodiments, sequence identity is determined across the entire length of a sequence, while in other embodiments, sequence identity is determined over a region of a sequence. “Identity” can also refer to the degree of sequence relatedness between two sequences as determined by the number of matches between strings of two or more residues (e.g., amino acid residues). Identity measures the percent of identical matches between the smaller of two or more sequences with gap alignments (if any) addressed by a particular mathematical model, algorithms, or computer program.

It will be appreciated that when a sequence of a first, shorter length is aligned with a sequence of a second, longer length, the resultant alignment may contain gaps in the first sequence that account for the relative difference in length between the two sequences. An “aligned sequence” or the “alignment of” a sequence, relative to another sequence, may include gaps or spaces, as necessary for the alignment of interest.

The identity of related polypeptide sequences can be readily calculated by any of the methods known to one of ordinary skill in the art. In some embodiments, the “percent identity” of two sequences (e.g., amino acid sequences) is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. Where gaps exist between two sequences, Gapped BLAST can be utilized, for example, as described in Altschul et al., Nucleic Acids Res. 25(17):3389-3402, 1997. When utilizing BLAST and Gapped BLAST programs, the default parameters of the respective programs (e.g., XBLAST and NBLAST) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences.

For multiple sequence alignments, computer programs including Clustal Omega (Sievers et al., Mol Syst Biol. 2011 Oct. 11; 7:539) may be used. In some embodiments, an amino acid sequence is found to have a specified percent identity to a reference sequence, such as a sequence disclosed in this application and/or recited in the claims, when sequence identity is determined using Clustal Omega (Sievers et al., Mol Syst Biol. 2011 Oct. 11; 7:539).

### Recombinant AAV Particle Compositions

Provided herein is a composition comprising any one of the recombinant AAVrh.10 or AAV3 particles disclosed herein.

In some embodiments, any one of the compositions provided herein comprises a pharmaceutically acceptable carrier. The term “carrier” refers to a diluent, adjuvant, excipient, or vehicle with which the rAAV particle is administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Delivering a Protein of Interest to a Subject

Subjects previously treated with AAV particles develop neutralizing antibodies against those particles. In such instances, treating the subject again with AAV particles that would react or even cross-react with the developed neutralizing antibodies results in an unwanted antigenic response. Any one of the compositions comprising any one of the variant AAVrh.10 or AAV3 particles that can escape neutralizing antibodies, as described herein, would be useful as a vehicle for gene delivery in such instances. Accordingly, provided herein is a method of delivering a protein of interest to a subject, the method comprising administering to the subject a composition comprising any one of the AAVrh.10 or AAV3 particles disclosed herein. Such a method diminishes the immunogenic response to the administered AAVrh.10 or AAV3 particles used to deliver a therapeutic gene.

Provided herein is also a method of reducing the antigenic response to AAVrh.10 or AAV3 particles administered to a subject. In some embodiments, the antigenic response to any one of the variant AAVrh.10 or AAV3 particles disclosed herein decreases by 5-100% (e.g., 5-100, 5-10, 10-30, 20-50, 20-70, 50-100, 5-60, 20-80 or 80-100%) compared to the antigenic response to a wild-type AAVrh.10 or AAV3 particle in the same or same type of subject. A same type of subject may have the same neutralizing antibodies.

As used herein, “administering” or “administration” means providing a material to a subject in a manner that is pharmacologically useful. In some embodiments, a subject is a mammal. In some embodiments, a mammalian subject is a human, a non-human primate, or a non-primate mammal, such as a dog, a cat, a hamster, a mouse, a rat, a pig, a horse, a cow, a donkey or a rabbit. In some embodiments, the subject is a human.

In certain circumstances it will be desirable to deliver the rAAV particles in suitably formulated pharmaceutical compositions disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection.

In some embodiments, a rAAVrh.10 or rAAV3 particle as described herein is administered to a subject to treat a disease or disorder. To “treat” a disease or disorder, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAVrh.10 or rAAV3 particles may be an amount of the particles that is capable of transferring an expression construct to a host organ, tissue, or cell. A therapeutically acceptable amount may be an amount that is capable of treating a disease, e.g., a hemophilia. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, the concentration of rAAV particles administered to a subject may be on the order ranging from 10⁶ to 10¹⁴ particles/ml or 10³ to 10¹⁵ particles/ml, or any values therebetween for either range, such as for example, about 10³, 10⁴, 10⁵, 10⁶, 10⁷, 10⁸, 10⁹, 10¹⁰, 10¹¹, 10¹², 10¹³, 10¹⁴, or 10¹⁵ particles/ml. In some embodiments, rAAV particles of a higher concentration than 10¹³ particles/ml are administered. In some embodiments, rAAV particles of a higher concentration than 10¹⁵ particles/ml are administered. In some embodiments, the concentration of rAAV particles administered to a subject may be on the order ranging from 10⁶ to 10¹⁴ vector genomes(vgs)/ml or 10³ to 10¹⁵ vgs/ml, or any values therebetween for either range (e.g., 10³, 10⁴, 10⁵, 10⁶, 10⁷, 10⁸, 10⁹, 10¹⁰, 10¹¹, 10¹², 10¹³, 10¹⁴, or 10¹⁵ vgs/ml). In some embodiments, rAAV particles of higher concentration than 10¹³ vgs/ml are administered. In some embodiments, rAAV particles of a higher concentration than 10¹⁵ particles/ml are administered. The rAAV particles can be administered as a single dose, or divided into two or more administrations as may be required to achieve therapy of the particular disease or disorder being treated. In some embodiments, 0.0001 ml to 10 ml are delivered to a subject. In some embodiments, the number of rAAV particles administered to a subject may be on the order ranging from 10⁶-10¹⁴ vg/kg, or any values therebetween (e.g., 10⁶, 10⁷, 10⁸, 10⁹, 10¹⁰, 10¹¹, 10¹², 10¹³, or 10¹⁴ vgs/mg). In some embodiments, the dose of rAAV particles administered to a subject may be on the order ranging from 10¹²-10¹⁴ vgs/kg. In some embodiments, the volume of rAAVrh.10 or rAAV3 composition delivered to a subject (e.g., via one or more routes of administration as described herein) is 0.0001 ml to 10 ml.

In some embodiments, a composition disclosed herein (e.g., comprising a rAAVrh.10 or rAAV3 particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject. In some embodiments, administration of a rAAVrh.10 or rAAV3 particle to a subject results in the prevention, alleviation or amelioration of one or more signs or symptoms of a disease or disorder.

In some embodiments, the subject has or is suspected of having a disease or disorder that may be treated with gene therapy. Non-limiting examples of conditions for which rAAV-based gene therapy may find particular utility include, but are not limited to, cancer, diabetes, autoimmune disease, kidney disease, cardiovascular disease, pancreatic disease, intestinal disease, liver disease, hemophilia, α1-antitrypsin (AAT) deficiency, ischemia, skeletal disease and pulmonary disease. In some embodiments, the disease or disorder is selected from the group consisting of: Wilson Disease, Glycogen Storage Disease Type Ia, Ornithine Transcarbamylase Deficiency, liver fibrosis, Hemophilia A, Hemophilia B, and Hemophilia C. In some embodiments, the disease or disorder is a cancer. In some embodiments, the cancer is one or more of cancer of the buccal cavity or pharynx, cancer of the digestive tract, cancer of the colon, rectum, or anus, cancer of the respiratory tract, breast cancer, cancer of the cervix, uteri, vagina, or vulva, cancer of the uterine corpus or ovary, cancer of the male genital tract, cancer of the urinary tract, bone or soft tissue cancer, Kaposi sarcoma, melanoma of the skin, ocular melanoma, non-melanoma eye cancer, cancer of the brain and central nervous system, cancer of the thyroid and other endocrine glands, Hodgkin Lymphoma, Non-Hodgkin Lymphoma, myeloma, renal cancer, colorectal cancer, lung cancer, breast cancer, pancreatic cancer, prostate cancer, gastric cancer, GIST or glioblastoma.

In some embodiments, the method comprises contacting a cell with a rAAVrh.10 or rAAV3 particle as described herein. In some embodiments, a cell disclosed herein is a cell isolated or derived from a subject. In some embodiments, a cell is a mammalian cell (e.g., a cell isolated or derived from a mammal). In some embodiments, the cell is a human cell, non-human primate cell, rat cell, or mouse cell. In some embodiments, a cell is isolated or derived from a particular tissue of a subject, such as liver tissue. In some embodiments, the cell is a liver, brain, heart or retina cell. In some embodiments, a cell is a liver cell. In some embodiments, a cell is in vitro. In some embodiments, a cell is ex vivo. In some embodiments, a cell is in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

Methods of contacting a cell may comprise, for example, contacting a cell in a culture with a rAAVrh.10 or rAAV3 particle as described herein. In some embodiments, contacting a cell comprises adding a rAAVrh.10 or rAAV3 particle as described herein to the supernatant of a cell culture (e.g., a cell culture on a tissue culture plate or dish) or mixing a rAAVrh.10 or rAAV3 particle as described herein with a cell culture (e.g., a suspension cell culture). In some embodiments, contacting a cell comprises mixing a rAAVrh.10 or rAAV3 particle as described herein with another solution, such as a cell culture media, and incubating a cell with the mixture.

In some embodiments, contacting a cell with a rAAVrh.10 or rAAV3 particle as described herein comprises administering a rAAVrh.10 or rAAV3 particle as described herein to a subject or device in which the cell is located. In some embodiments, contacting a cell comprises injecting a rAAVrh.10 or rAAV3 particle as described herein into a subject in which the cell is located. In some embodiments, contacting a cell comprises administering a rAAVrh.10 or rAAV3 particle as described herein directly to a cell, or into or substantially adjacent to a tissue of a subject in which the cell is present.

Vectors and Kits Useful in the Preparation of rAAV Particles

In some embodiments, provided herein is a vector comprising a nucleic acid encoding one or more AAVrh.10 or AAV3 Cap proteins with any one or more of the mutations described herein. Such a vector can be used to prepare or package any one of the AAVrh.10 or AAV3 particles disclosed herein. In some embodiments, provided herein is a vector comprising a nucleic acid encoding one or more genes of interest and/or one or more promoters. In some embodiments, a vector is a plasmid. In some embodiments, a vector (e.g., a plasmid) is provided in a host cell.

Methods of producing rAAV particles are known in the art (see, e.g., Zolotukhin, et al., Methods (2002); 28(2):158-67; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and transfected into recombinant cells such that the recombinant AAV particle can be packaged and subsequently purified.

In some embodiments, the one or more helper plasmids include a first helper plasmid comprising a rep gene, a nucleic acid encoding any one of the Cap proteins disclosed herein, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA.

In some embodiments, the packaging is performed in a helper cell or producer cell, such as a mammalian cell or an insect cell. Non-limiting examples of mammalian cells are HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). A non-limiting example of an insect cell is Sf9 cells (see, e.g., ATCC® CRL-1711™). A helper cell may comprise rep genes that encode the Rep proteins for use in a method described herein. In some embodiments, the packaging is performed in vitro.

Provided herein is also a kit that comprises tools for the preparing of any one of the AAVrh.10 or AAV3 particles provided herein. Such a kit comprises any one of the vectors encoding any one of the AAVrh.10 or AAV3 Cap proteins described herein, as a first component. A kit may also comprise a vector comprising AAV helper genes as a second component, wherein each component is packaged in separate containers. In some embodiments, a kit also comprises AAV packaging cells that are provided in a third container that is separate from the first and second containers.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Enumerated Embodiments

Clause 1. A recombinant adeno-associated virus 3 (rAAV3) particle comprising a capsid protein comprising one or more mutations, wherein said mutations result in modulated reactivity to a neutralizing antibody and/or altered transduction efficiency relative to a wild-type AAV3 particle having a capsid protein with an amino acid sequence of SEQ ID NO: 24 or SEQ ID NO: 26.
Clause 2. The rAAV3 particle of clause 1, wherein the rAAV3 particle is a rAAV3a particle or a rAAV3b particle.
Clause 3. The rAAV3 particle of clause 1 or clause 2, wherein the neutralizing antibody is against AAV3.
Clause 4. The rAAV3 particle of clause 1 or clause 2, wherein the neutralizing antibody is against AAV1, AAV4, AAV5, AAV6, AAV7, AAV9, AAV10, AAVrh.10, AAV11, AAV12, AAV13, or AAVrh.39.
Clause 5. The rAAV3 particle of clause 1 or clause 2, wherein the neutralizing antibody is against AAV2 or AAV8.
Clause 6. The rAAV3 particle of clause 1 or clause 2, wherein the neutralizing antibody is ADK8, IVIG, HL2381 or HL2383.
Clause 7. The rAAV3 particle of clause 6, wherein the neutralizing antibody is ADK8.
Clause 8. The rAAV3 particle of clause 6, wherein the neutralizing antibody is IVIG.
Clause 9. The rAAV3 particle of clause 6, wherein the neutralizing antibody is HL2381.
Clause 10. The rAAV3 particle of clause 6, wherein the neutralizing antibody is HL2383.
Clause 11. The rAAV3 particle of any of the preceding clauses, wherein the reactivity to neutralizing antibodies is decreased compared to wild-type AAV3 particles.
Clause 12. The rAAV3 particle of clause 11, wherein the reactivity to neutralizing antibodies is decreased by 50-100% compared to wild-type AAV3 particles.
Clause 13. The rAAV3 particle of clause 11 or 12, wherein the reactivity to neutralizing antibodies is decreased by 75-100% compared to wild-type AAV3 particles.
Clause 14. The rAAV3 particle of any one of the preceding clauses, wherein the capsid protein is one or more of the capsid proteins selected from the group consisting of: VP1, VP2 and VP3.
Clause 15. The rAAV3 particle of any one of the preceding clauses, wherein the one or more mutations comprise an amino acid substitution or an amino acid deletion.
Clause 16. The rAAV3 particle of any one of the preceding clauses, wherein the one or more mutations are at amino acid positions selected from the group consisting of: N588, A590, S384, and T717.
Clause 17. The rAAV3 particle of any one of the preceding clauses, wherein the one or more mutations are amino acid substitution(s) selected from the group consisting of: N588A, N588S, A590Q, S384A, and T717V.
Clause 18. The rAAV3 particle of clause 17, wherein the one or more mutations is N588A.
Clause 19. The rAAV3 particle of clause 17, wherein the one or more mutations is N588S.
Clause 20. The rAAV3 particle of clause 17, wherein the one or more mutations is A590Q.
Clause 21. The rAAV3 particle of clause 17, wherein the one or more mutations is S384A.
Clause 22. The rAAV3 particle of clause 17, wherein the one or more mutations is T717V.
Clause 23. The rAAV3 particle of clause 17, wherein the one or more mutations are N588A and A590Q.
Clause 24. The rAAV3 particle of clause 17, wherein the one or more mutations are N588S and A590Q.
Clause 25. The rAAV3 particle of clause 17, wherein the one or more mutations are N588A, A590Q, S384A, and T717V.
Clause 26. The rAAV3 particle of clause 17, wherein the one or more mutations are N588S, A590Q, S384A, and T717V.
Clause 27. The rAAV3 particle of any one of the preceding clauses, further comprising a transgene comprising a gene of interest.
Clause 28. The rAAV3 particle of clause 27, wherein the gene of interest encodes a therapeutic protein.
Clause 29. The rAAV3 particle of clause 28, wherein the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.
Clause 30. The rAAV3 particle of clause 27 or clause 28, wherein the gene of interest encodes a detectable molecule.
Clause 31. The rAAV3 particle of clause 30, wherein the detectable molecule is a fluorescent protein, a bioluminescent protein, or a protein that provides color, or a fragment thereof.
Clause 32. A composition comprising a rAAV3 particle of any one of the preceding clauses.
Clause 33. The composition of clause 32, further comprising a pharmaceutically acceptable carrier.
Clause 34. A method of delivering a protein of interest to a subject, the method comprising administering to the subject a composition comprising a rAAV3 particle of any one of clauses 27-33, wherein the gene of interest encodes a protein of interest.
Clause 35. The method of clause 34, wherein the protein of interest is a therapeutic protein or detectable molecule.
Clause 36. The method of clause 35, wherein the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant.
Clause 37. The method of clause 35 or clause 36, wherein the detectable molecule is a fluorescent protein, a bioluminescent protein, or a protein that provides color, or a fragment thereof.
Clause 38. The method of any one of clauses 34-37, wherein the subject is a mammalian subject.
Clause 39. The method of clause 38, wherein the mammalian subject is a human, a non-human primate, non-primate mammal, or mouse.
Clause 40. The method of any one of clauses 34-39, wherein the subject has or is suspected of having a disease or disorder comprising cancer, diabetes, autoimmune disease, kidney disease, cardiovascular disease, pancreatic disease, intestinal disease, liver disease, hemophilia, α1-antitrypsin (AAT) deficiency, ischemia, skeletal disease and pulmonary disease.
Clause 41. The method of clause 40, wherein administration of the rAAV3 particle to the subject results in the prevention, alleviation or amelioration of one or more signs or symptoms of the disease or disorder.
Clause 42. The method of any one of clauses 34-41, wherein the rAAV3 particle is administered to the subject subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs of the subject.
Clause 43. A vector comprising a nucleic acid encoding Cap proteins, wherein the Cap proteins form a rAAV3 particle of any one of clauses 1-33 when the vector is used to form capsids.
Clause 44. A kit comprising the vector of clause 43 and a vector comprising AAV helper genes, wherein the vector of clause 43 and the vector comprising AAV helper genes are provided in a first and second container, wherein the first and second containers are different.
Clause 45. The kit of clause 44, further comprising AAV packaging cells that are provided in a third container that is separate from the first and second containers.
Clause 46. The kit of clause 44 or clause 45, wherein the AAV helper genes encode E1, E2, E4 and/or VA helper proteins.
Clause 47. A capsid protein of serotype AAV3 comprising one or more mutations in amino acid positions selected from the group consisting of: N588 and A590 in SEQ ID NO: 24 or SEQ ID NO: 26.
Clause 48. The capsid protein of clause 47, wherein the one or more mutations are amino acid substitution(s) selected from the group consisting of: N588A, N588S, and A590Q.
Clause 49. The capsid protein of clause 47 or clause 48, wherein the one or more mutations are N588A and A590Q.
Clause 50. The capsid protein of clause 47 or clause 48, wherein the one or more mutations are N588A and A590Q.
Clause 51. A capsid protein of serotype AAV3 comprising one or more mutations in amino acid positions selected from the group consisting of: N588, A590, S384, and T717 in SEQ ID NO: 24 or SEQ ID NO: 26.
Clause 52. The capsid protein of clause 51, wherein the one or more mutations are amino acid substitution(s) selected from the group consisting of: N588A, N588S, A590Q, S384A, and T717V.
Clause 53. The capsid protein of clause 51 or clause 52, wherein the one or more mutations are N588A, A590Q, S384A, and T717V.
Clause 54. The capsid protein of clause 51 or clause 52, wherein the one or more mutations are N588S, A590Q, S384A, and T717V.
Clause 55. A recombinant AAV3 particle comprising the capsid protein of any one of clauses 47-54.

### EXAMPLES

### Example 1

### Solving the Structure of AAVrh.10 Particles

### Structural Analysis

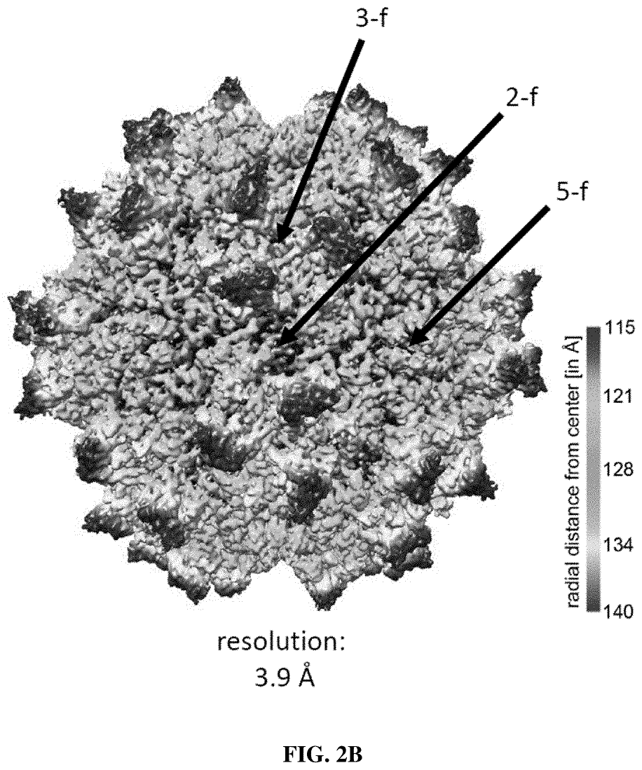

As mentioned above, information about the structure of AAVrh.10 particles was not previously known. To this end, cryo-electron microscopy (cryo-EM) and image reconstruction was therefore used to solve the structure of AAVrh.10 capsids. FIG. 2A and FIG. 2B show the surface topology of the solved structure. It was observed that AAVrh.10 exhibits the surface topology conserved in all AAVs, including depressions at the 2-fold axis, cylindrical channel at the 5-fold axis, and three protrusions around the 3-fold axis.

Thereafter, a structural alignment was performed comparing the capsid protein VP3 of AAVrh.10 to VP3 of AAV8, which has 92.5% sequence identity to VP3 of AAVrh.10. The AAV8 VP3 information can be found in the protein data base (PDB#: 3RA2). FIG. 3A shows the alignment of AAVrh.10 VP3 to AAV8 VP3.

A more thorough analysis was then performed on the VR VIII loop as shown in FIGS. 3A and 3C, and which contains the biding epitope on AAV8 to neutralizing antibody ADK8. It can be seen in FIG. 3C that AAVrh.10 aligns quite well with AAV8 in this region.

### Antibody Recognition

Given the close alignment of AAV8 and AAVrh.10 (FIG. 3C), experiments were conducted to assess the antibody recognition of AAVrh.10 by antibodies known to react with AAV8. FIG. 4 shows that antibodies against AAV8 (e.g., ADK8, ADK8/9, HL2381 and HL2383) cross-react with AAVrh.10.

### Summary

The experimental data described in this example provides the basis for designing and engineering AAVrh.10 variants with mutations that might decrease the cross-reactivity to neutralizing antibodies.

### Example 2

### Generation of AAVrh.10 Antibody Escape Mutants

Based on the structural information described in Example 1, numerous mutations in AAVrh.10 capsid proteins were designed and tested for binding to cross-reacting neutralizing antibodies (FIG. 5A).

FIG. 5B and FIG. 5C show the amino acid sequence of the ADK8 and ADK8/9 epitopes, respectively. Based on the conserved residues among AAV serotypes that bind and do not bind to ADK8, mutations were made at positions N590 and A592 (FIG. 5B). Based on the conserved residues among AAV serotypes that bind and do not bind to ADK8/9, an additional mutation were made at position Q589 (FIG. 5C). FIGS. 5D-5F show the antibody escape phenotype variant AAVrh.10 particles with single and multiple mutations made in AAVrh.10. It was found that the variant AAVrh.10 with N590S andA592Q mutations has reduced reactivity to antibodies ADK8, ADK8/9 and HL2383 (FIG. 5D and FIG. 5E). AAVrh.10 with mutations Q589N, N590S and A592Q also showed diminished reactivity to ADK8 and ADK8/9 (FIG. 5F).

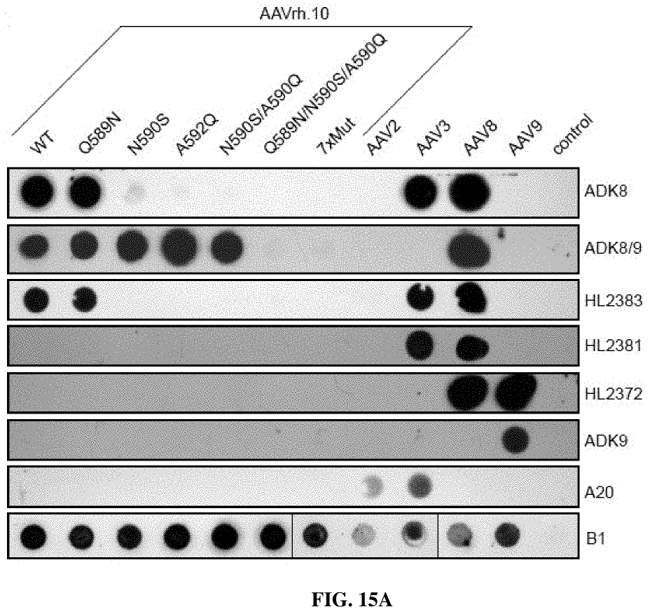

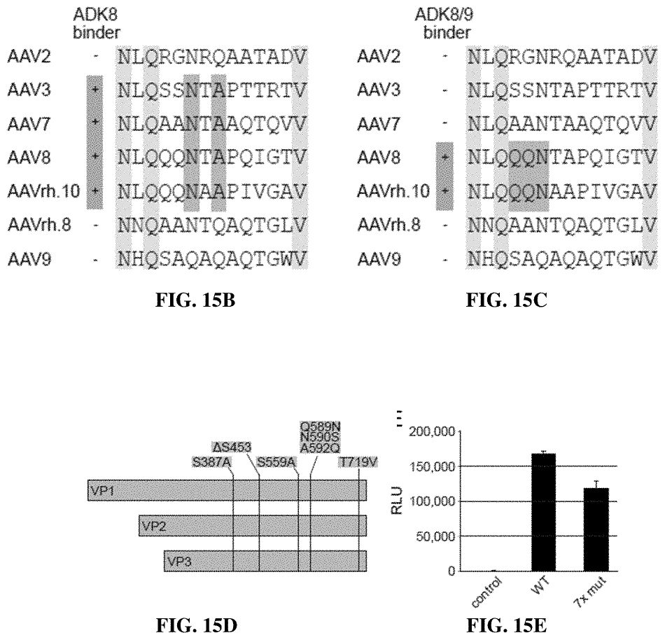

Further determination of the antigenicity of the AAVrh.10 capsid is shown in FIG. 15. FIG. 15A shows immune-dot blot analysis of native rAAV capsids of indicated serotypes. 10¹⁰ genome-containing particles were spotted on a nitrocellulose membrane. The membranes were incubated with a panel of mAbs as depicted to the left of the membrane. Monoclonal antibody B1 served as an internal loading control. FIGS. 15B and 15C show sequence alignments for the VR-VIII loop of a selection of AAV serotypes (SEQ ID NOs: 16-22 from top to bottom, respectively). Amino acids highlighted in light grey indicate sequence identity among the AAVs. In addition, amino acids highlighted in darker grey indicate common residues among the ADK8 binding AAV serotypes in the ADK8 binding epitope (FIG. 15B) and among the ADK8/9 binding AAV serotypes in the ADK8/9 binding epitope (FIG. 15C). FIG. 15D shows the relative positions of seven mutations (7× mut) introduced into the VP proteins of AAVrh.10 are shown. FIG. 15E shows a comparison of the transduction of AAVrh.10 wild-type vectors to the generated AAVrh.10 7× mutant in HEK 293 cells by a luciferase assay (MOI 100,000).

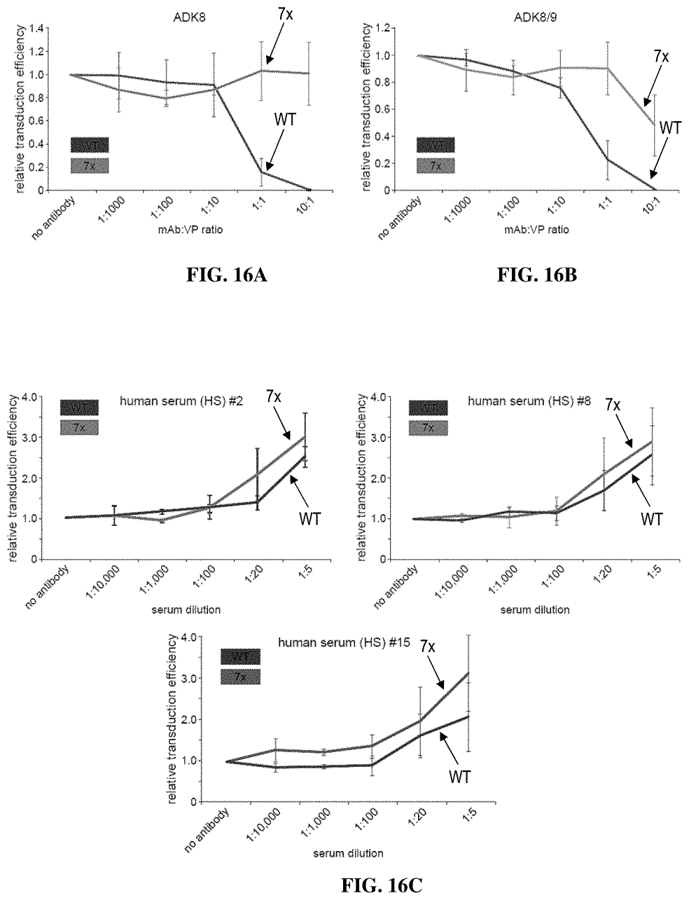

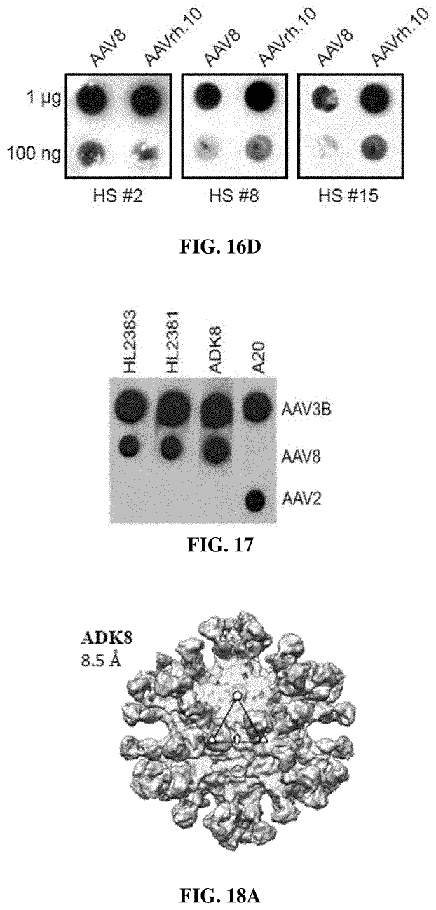

Prevention of antibody-mediated AAVrh.10 neutralization is shown in FIG. 16. FIGS. 16A and 16B show a neutralization assay using increasing amounts of purified monoclonal antibodies, ADK8 in (FIG. 16A) and ADK8/9 in (FIG. 16B) with purified AAVrh.10 wild-type (dark grey) or 7× mut (light grey) vectors carrying a luciferase gene (MOI 100,000). FIG. 16C shows a similar neutralization assay as in (FIG. 16A) except that different human serum samples were used instead of monoclonal antibodies. FIG. 16D shows native dot blot analysis using the human serum samples used in (FIG. 16C) as primary antibodies on AAV8 and AAVrh.10.

### Example 3

### Identification of AAVrh.10 Cellular Receptor and Receptor Binding Site on the Capsid Surface

As discussed above, very little is known about AAVrh.10, including what the cell-surface receptors are that are involved in transduction or infection of a cell by the AAVrh.10 particle. To gain some insight into this infection process, experiments were carried out to first tease out potential receptors, and then determine what the receptor binding site/s are on the AAVrh.10 capsid surface.

### Identification of AAVrh.10 Receptor

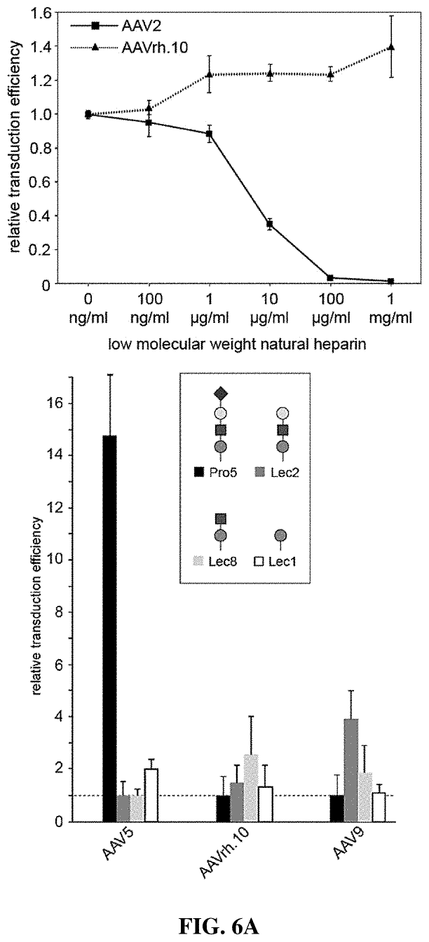

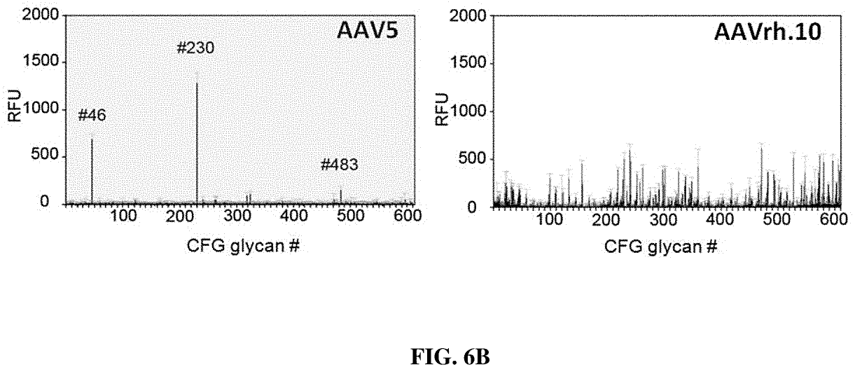

FIGS. 6A and 6B show that the receptor for AAVrh.10 is not a heparan sulphate proteoglycans (HSPG), sialic acid or a terminal galactose. Data in FIG. 6A were obtained by carrying out transduction of cells in the presence of molecules that would block potential receptors from binding to AAVrh.10.

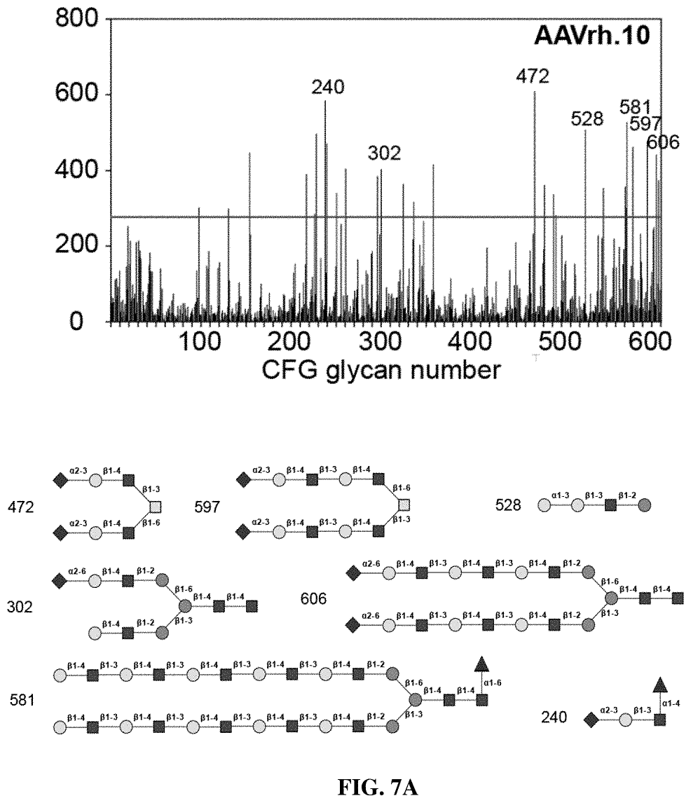

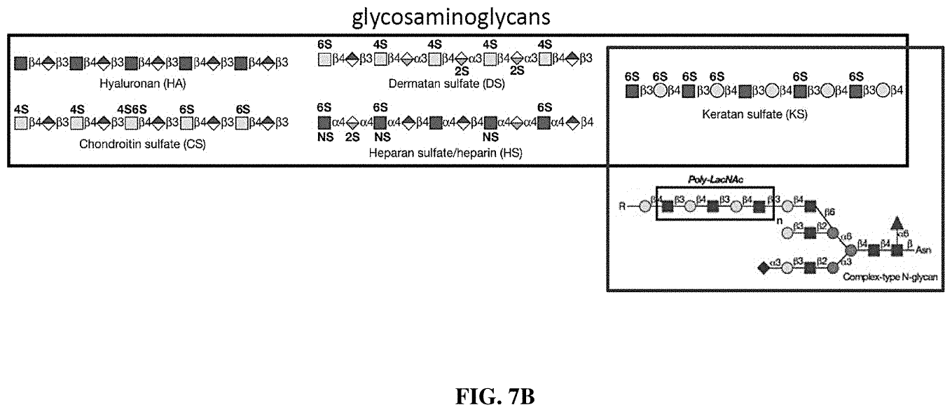

While a glycan array was initially found to be inconclusive (FIG. 6B), a closer analysis of data from a previously performed Consortium for Functional Glycomics (CFG) array, using a lower threshold for calling hits, found that AAVrh.10 bound to certain glycoaminoglycans, particularly those containing N-acetyllactosamine (LacNAc) (FIGS. 7A and 7B).

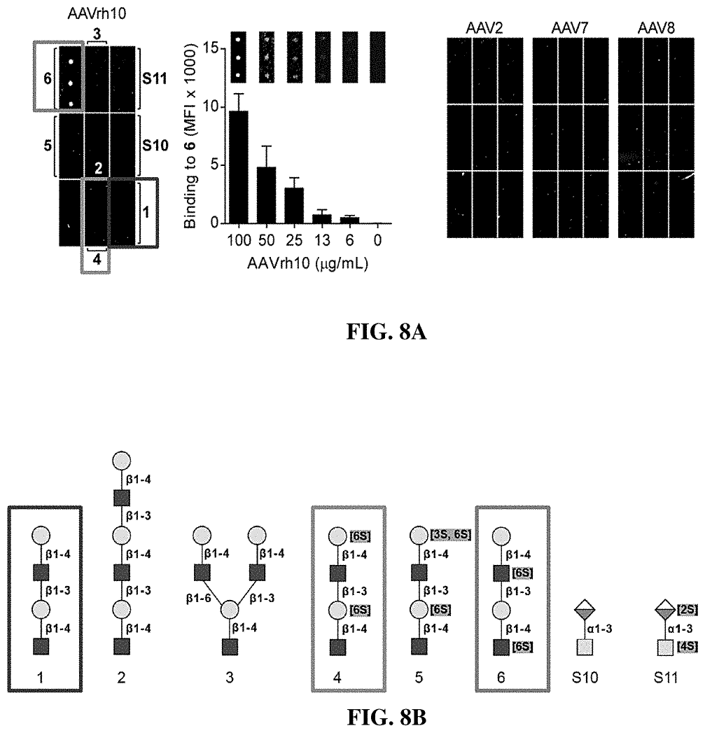

Upon further screening of AAVs on LacNAc glycan array, it was found that AAVrh.10 binds to sulfated LacNAc and that 6S N-acetyl-glucosamine is required for this binding (FIG. 8A and 8B).

### Identification of the Receptor Binding Site by Cryo-EM

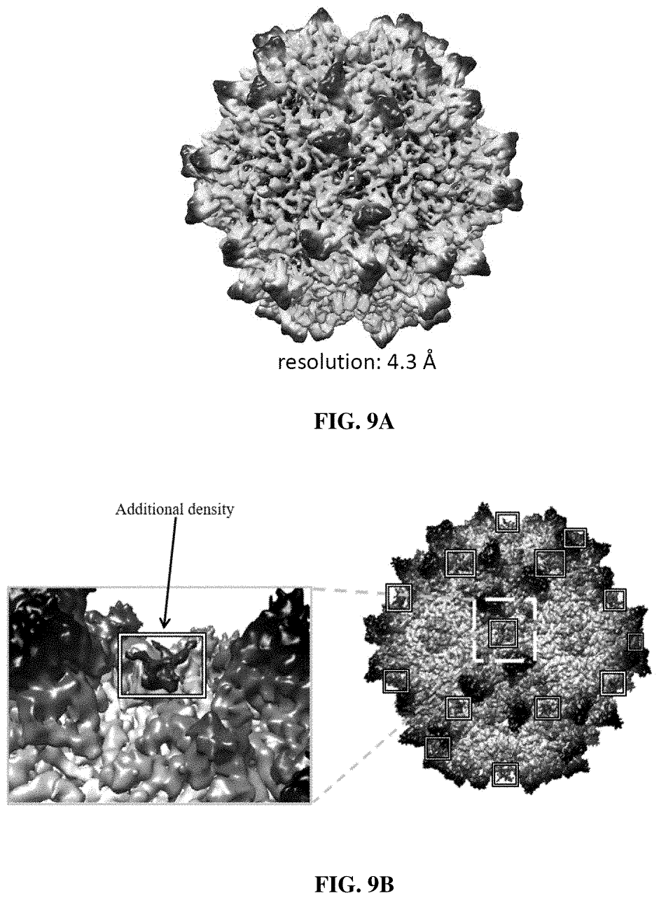

Cryo-EM and image reconstruction of complexed AAVrh.10 capsid with excess LacNAc glycan molecules (#6, ˜1 kDa) at a ratio of 100 glycan molecules per VP monomer was performed. FIG. 9A shows the cryo-EM reconstruction of AAVrh.10 complexed with LacNAc glycan. A subtraction was then carried out between the AAVrh.10 density map from the AAVrh.10 complexed with glycan to uncomplexed AAVrh.10. FIG. 9B shows the resultant difference map revealing additional density at the surface of the 2-fold symmetry axis.

Thereafter, an analysis of the conserved amino acids in the identified region was performed to determine amino acid residues necessary for receptor binding (FIG. 10A). It was found that Y708 was the only residue that is not conserved in all AAV serotypes (FIG. 10B). In further experiments testing cell transduction, it was observed that a Y708A mutation lead to reduced transduction of cells as assessed by transduction and expression of luciferase (FIG. 10C).

### Effect of AAVrh.10 Mutations on Transduction Efficiency

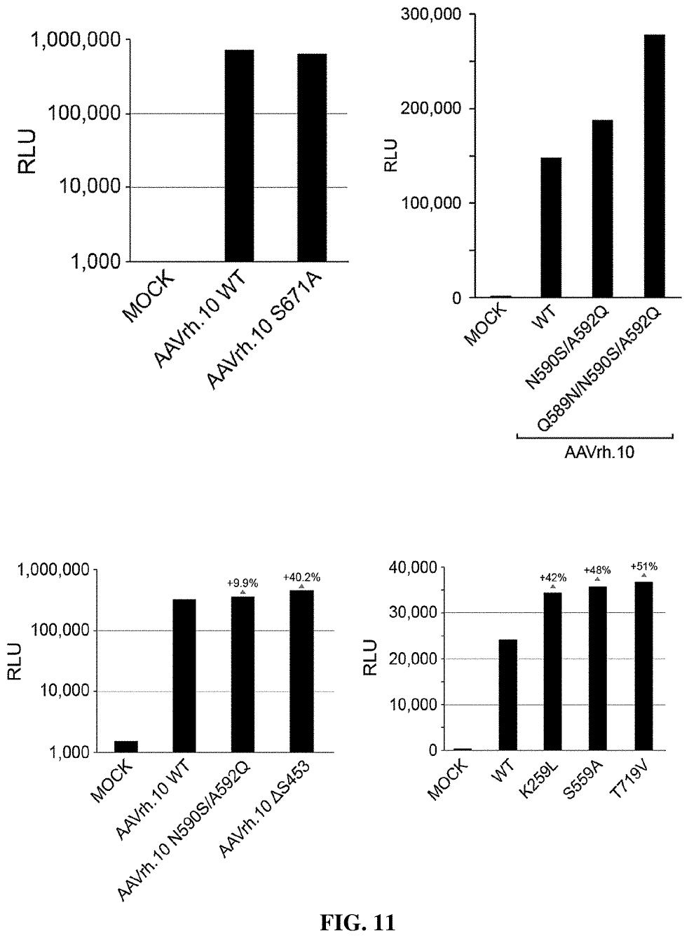

FIG. 11 shows that all variant AAVrh.10 particles tested did not show diminished transduction efficiency, and in fact, most, with the exception of S671A, showed increased transduction efficiency. Cells were transduced with wild-type and engineered variant AAVrh.10 particles comprising a gene encoding luciferase.

### Summary

The information of which capsid surface region and which amino acid residues are necessary for binding to LacNAc receptors can be used to avoid generating mutations that would detrimentally affect cell transduction by variant AAVrh.10 particles compared to wild-type AAVrh.10 particles.

### Example 4

### AAV3b Variants

A structure-guided approach was used to develop AAV3b variants with host antibody escaping abilities.

Neutralizing antibody (Nab) binding to the AAV3b capsid can reduce transduction of liver cells. Therefore, AAV3b capsid variants capable of antibody escape could increase vector efficacy, particularly for delivery of therapeutic molecules to the liver. Like with AAVrh.10, several AAV8 monoclonal antibodies cross-react to the AAV3b capsid, including HL2381, HL2383, ADK8. The antibody IVIG may also cross-react to the AAV3b capsid. Based on the structural similarity between AAV2 and AAV3 (e.g., AAV3b), neutralizing antibodies against AAV2, such as the antibody A20, may also cross react to the AAV3b capsid. For example, FIG. 17 shows cross-reactivity between conformation-specific monoclonal antibodies raised against the AAV2 capsid, the AAV8 capsid and the AAV3b capsid, implying structural conservation of some epitopes among the capsid serotypes.

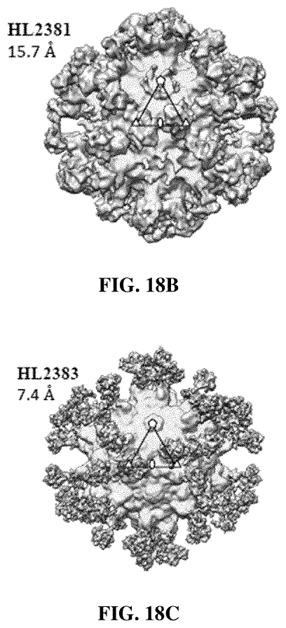

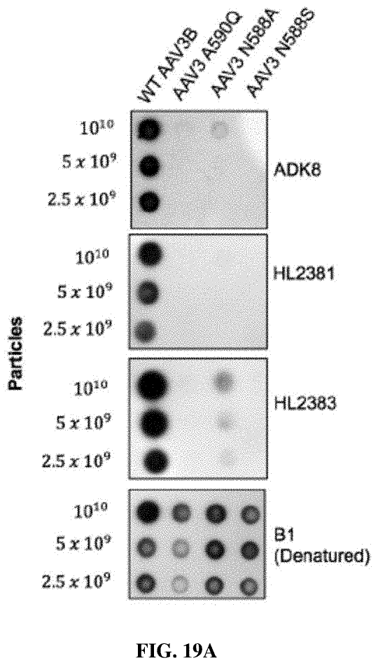

Pseudoatomic models of binding between the virus and antigen-binding fragments (Fabs) were developed. FIGS. 18A-18C show 3D cryo-reconstructions of purified AAV3b wild-type capsids in complex with purified Fabs. The reconstructions demonstrate that ADK8, HL2381, and HL2383 all share an antigenic site atop the 3-fold viral protrusions. The AAV3b amino acid residues involved in Fab binding for all three antibodies (ADK8, HL2381, HL2383) are all located in the variable region VIII. These reconstructions and the AAV3b amino acid residues involved in Fab binding for ADK8, HL2381, HL2383 served as the bases for the rational design of AAV3b antibody escape variants.

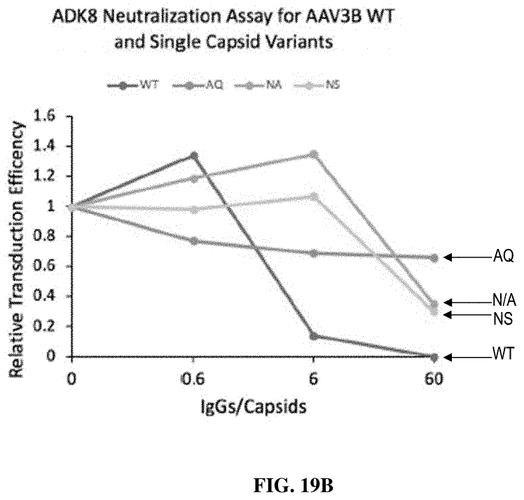

To confirm the contact residues of the antibodies on the capsid, single amino acid AAV3b capsid variants A590Q, N588A, and N588S were produced, purified, and tested by dot blot and neutralization assay for cross reactivity with HL2381, HL2383, and ADK8 antibodies. The dot blots of FIG. 19A show that the recognition of the capsid variants (e.g., the A590Q, N588A, and N588S single mutation AAV3b variants) by the three anti-AAV8 antibodies (A590Q, N588A, and N588S) is reduced compared to wild-type AAV3b. Though recognition of the N588A variant was reduced compared to the wild-type capsid, weak detection of the N588A variant was still observed. The neutralization assay of FIG. 19B demonstrates that the infectivity of the AAV3b capsid variants (e.g., the A590Q, N588A, and N588S single mutation AAV3b variants) in presence of the A590Q, N588A, and N588S antibodies showed a slight reduction of transgene expression for all samples.

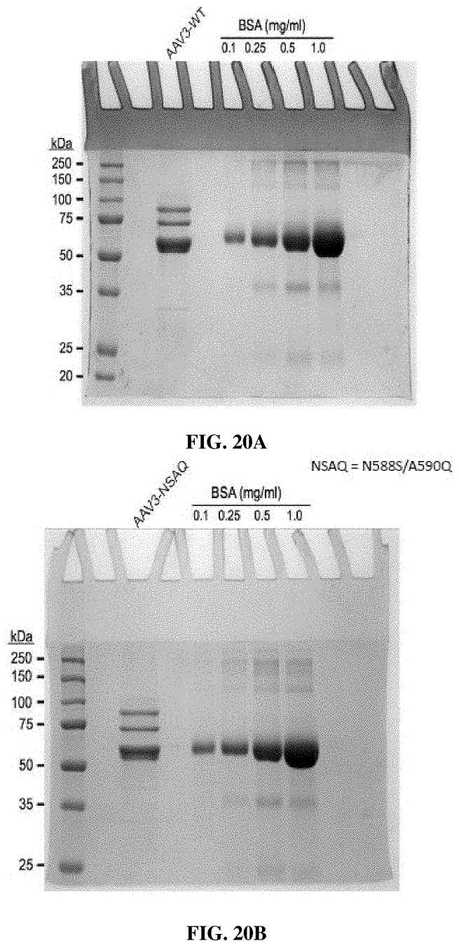

An AAV3b variant comprising the mutations N588S and A590Q was developed based on analysis of cross-reactivity antigenic epitopes that escapes recognition by a series of cross-reactive antibodies while maintaining wild-type infectivity (termed “NSAQ”). The production of wild-type AAV3b and the AAV3b variant NSAQ yielded highly pure and high titer samples. As shown in FIG. 20A, the wild-type AAV3b production was estimated to be ˜0.6 mg/ml. Subsequent qPCR analysis determined the viral titer for the wild-type AAV3b particles to be 3.75×10¹³ genome particles/ml. FIG. 20B shows that production of the AAV3b variant NSAQ was estimated to also be ˜0.6 mg/ml. Subsequent qPCR analysis determined the viral titer for the AAV3b variant NSAQ to be 1.265×10¹³ genome particles/ml.

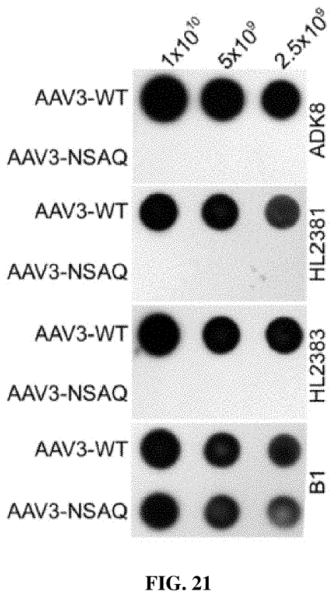

Dot blots were performed to verify antibody binding to the AAV3b wild-type capsid and determine if the AAV3b variant NSAQ variant was capable of antibody evasion. The antibodies ADK8, HL2381, and HL2383 were used. B1 was used as a loading control. The dot blot results confirm that wild-type AAV3b was cross-reactive with all three anti-AAV8 antibodies (ADK8, HL2381, and HL2383). FIG. 21 demonstrates that the AAV3b variant NSAQ exhibits full antibody escape for the anti-AAV8 antibodies (ADK8, HL2381, and HL2383) at all concentrations tested.

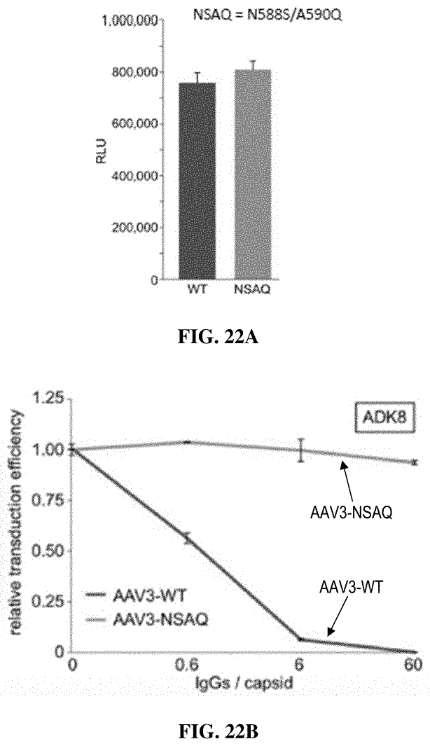

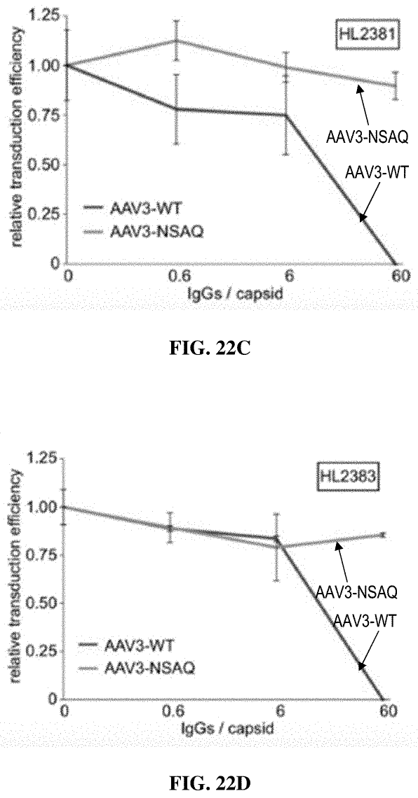

Experiments were conducted to verify that the AAV3b variant NSAQ transduces cells in the presence of neutralizing antibodies. Each AAV capsid has 60 antibody binding sites due to its icosahedral symmetry (1:1 (60 IgGs per capsid); 1:10 (6 IgGs per capsid); 1:100 (0.6 IgGs per capsid)). Accordingly, purified, full-length IgG antibodies were used to ensure a quantified number of antibodies were incubated with virus at the correct ratio. FIGS. 22A-22D show that the AAV3b variant NSAQ shows similar transduction efficiency to a wild-type AAV3b capsid in the absence (FIG. 22A) and presence (FIGS. 22B-22D) of neutralizing antibodies. As can be seen in FIG. 22B, the antibody ADK8 neutralized the wild-type AAV3b capsid most effectively. However, the neutralization assay results (FIGS. 22B-D) agree with the dot blot results (FIG. 21) that the AAV3b variant NSAQ exhibits total antibody escape for the anti-AAV8 antibodies ADK8, HL2381, and HL2383.

The results above describe non-limiting examples of certain AAV3b variants, and other AAV3 variants may be developed. For example, an AAV3b variant comprising the mutations N588A and A590Q is developed. An alignment of the AAVrh.10 and AAV3b capsids was performed. Candidate mutations in the AAVrh.10 capsid, as described herein, were mapped onto the AAV3b capsid. Based on this alignment, one or more AAV3b variants comprising the additional mutations S384A and T717V is developed. Said AAV3b variant may comprise, for example, the mutations N588A, A590Q, S384A, and T717V. Alternatively, said AAV3b variant may comprise, for example, the mutations N588S, A590Q, S384A, and T717V. Each of these AAV3b variants also exhibits total antibody escape from neutralizing antibodies, while maintaining or improving transduction efficiency, relative to a wild-type AAV3b capsid (FIG. 23).

AAV3b vectors are being developed for liver-targeted gene therapies. The modified AAV3b vectors described herein could be used in human gene delivery for patients that test positive for neutralizing antibodies against different AAV serotypes (especially AAV7, AAV8, and AAVrh.10) who would otherwise be excluded from clinical trials or treatment. AAV3b vectors with host antibody escaping properties, and without altered tissue targeting properties, are generated as described herein. These AAV3b vectors can be used in human gene therapy for patients who test positive for neutralizing antibodies against AAV3. Typically, patients who test positive against the AAV serotype of choice for a specific treatment are excluded from the cohort. Gene therapy vectors based on the AAV3b variants of the disclosure that escape from pre-existing antibodies will provide an alternative capsid for the treatment of seropositive patients.

An AAV3b gene therapy vector comprising an AAV3b capsid variant of the disclosure and a transgene comprising a gene of interest encoding a therapeutic protein is administered to a subject having a liver-related disease, such as hemophilia. Administration of said AAV3b gene therapy vector treats the liver-related disease (e.g., hemophilia) by alleviating one or more symptoms of the liver-related disease, such as, in the case of hemophilia, bleeding into the skin causing a hematoma. Confirmation of such alleviation may be confirmed visually by the attending physician or medical professional.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be illustrative examples, and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant adeno-associated virus 3 (rAAV3) particle comprising a capsid protein comprising one or more mutations, wherein said mutations result in modulated reactivity to a neutralizing antibody and/or altered transduction efficiency relative to a wild-type AAV3 particle having a capsid protein with an amino acid sequence of SEQ ID NO: 24 or SEQ ID NO: 26.

2. The rAAV3 particle of claim 1, wherein the rAAV3 particle is a rAAV3a particle or a rAAV3b particle.

3. The rAAV3 particle of claim 1, wherein the neutralizing antibody is against AAV1, AAV2, AAV3, AAV4, AAV5, AAV6, AAV7, AAV8, AAV9, AAV10, AAVrh.10, AAV11, AAV12, AAV13, or AAVrh.39.

4. The rAAV3 particle of claim 1, wherein the neutralizing antibody is ADK8, IVIG, HL2381, or HL2383.

5. The rAAV3 particle of claim 1, wherein the reactivity to neutralizing antibodies is decreased compared to wild-type AAV3 particles.

6. The rAAV3 particle of claim 5, wherein the reactivity to neutralizing antibodies is decreased by 50-100% or by 75-100% compared to wild-type AAV3 particles.

7. The rAAV3 particle of claim 1, wherein the capsid protein is one or more of the capsid proteins selected from the group consisting of: VP1, VP2 and VP3.

8. The rAAV3 particle of claim 1, wherein the one or more mutations comprise an amino acid substitution or an amino acid deletion.

9. The rAAV3 particle of claim 1, wherein the one or more mutations are at amino acid positions selected from the group consisting of: N588, A590, S384, and T717.

10. The rAAV3 particle of claim 1, wherein the one or more mutations are amino acid substitution(s) selected from the group consisting of: N588A, N588S, A590Q, S384A, and T717V.

11. The rAAV3 particle of claim 10, wherein the one or more mutations is/are:
(i) N588A;
(ii) N588S;
(iii) A590Q;
(iv) S384A;
(v) T717V;
(vi) N588A and A590Q;
(vii) N588S and A590Q;
(viii) N588A, A590Q, S384A, and T717V; or
(ix) N588S, A590Q, S384A, and T717V.

12. The rAAV3 particle of claim 1, further comprising a transgene comprising a gene of interest, wherein the gene of interest encodes a therapeutic protein or a detectable molecule.

13. The rAAV3 particle of claim 12, wherein:
(i) the therapeutic protein is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, or an anticoagulant; or
(ii) the detectable molecule is a fluorescent protein, a bioluminescent protein, or a protein that provides color, or a fragment thereof.

14. A composition comprising a rAAV3 particle of claim 1, and further comprising a pharmaceutically acceptable carrier.

15. A method of delivering a protein of interest to a subject, the method comprising administering to the subject a composition comprising a rAAV3 particle of claim 12, wherein the gene of interest encodes a protein of interest, wherein the protein of interest is a therapeutic protein or detectable molecule, and wherein the subject is a mammalian subject.

16. The method of claim 15, wherein the subject has or is suspected of having a disease or disorder selected from the group consisting of: cancer, diabetes, autoimmune disease, kidney disease, cardiovascular disease, pancreatic disease, intestinal disease, liver disease, hemophilia, α1-antitrypsin (AAT) deficiency, ischemia, skeletal disease and pulmonary disease and wherein administration of the rAAV3 particle to the subject results in the prevention, alleviation or amelioration of one or more signs or symptoms of the disease or disorder.

17. A vector comprising a nucleic acid encoding Cap proteins, wherein the Cap proteins form a rAAV3 particle of claim 1 when the vector is used to form capsids.

18. A kit comprising the vector of claim 17 and a vector comprising AAV helper genes, wherein the vector of claim 17 and the vector comprising AAV helper genes are provided in a first and second container, wherein the first and second containers are different.

19. A capsid protein of serotype AAV3 comprising one or more mutations in amino acid positions selected from the group consisting of:
(i) N588 and A590 in SEQ ID NO: 24 or SEQ ID NO: 26, wherein the one or more mutations are:
(a) N588A and A590Q; or
(b) N588S and A590Q; or
(ii) N588, A590, S384, and T717 in SEQ ID NO: 24 or SEQ ID NO: 26, wherein the one or more mutations are:
(a) N588A, A590Q, S384A, and T717V; or
(b) N588S, A590Q, S384A, and T717V.

20. A recombinant AAV3 particle comprising the capsid protein of claim 19.
